SEQUENCE LISTING (140 sequences)

SEQ ID NO: 1 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 1210 aa, Homo sapiens [sequence omitted]

SEQ ID NO: 3 - protein, 122 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 4 - protein, 120 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 5 - protein, 125 aa, synthetic construct
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 6 - protein, 119 aa, synthetic construct
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 7 - protein, 126 aa, synthetic construct
QVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 8 - protein, 126 aa, synthetic construct
QVQLQESGPGLVKPSETLSLTCTVSNGSISTYYWSWIRQPPGKGLEWIGYVYYTGRTKYNPSLKSRVTISVDTSKNQFSLNLSSVTAADTAVYYCARGGIVVVPAARDYYYYMDVWGKGTTVTVSS

SEQ ID NO: 9 - protein, 120 aa, synthetic construct
EVQLVQSGAEVKKPGESLKIACKGSGFSFTSHWIGWVRQKPGRGLEWMGVIYPGDSDTRYSPSFQGQVTVSADKSINTAYLQWNSLKASDTAIYYCARPNSGSPRYFEFWGRGTLVTVSS

SEQ ID NO: 10 - protein, 126 aa, synthetic construct
QVQLQESGPGLVKPSETLSLTCTVSGGSISSSSYYWGWIRQPPGKGLEWIGSFYYSGNTYYNPSLKSRVTISVDTSKNQFSLKLSSVTAADTAVYYCARQEYYYGSGSPSYYFDYWGQGTLVTVSS

SEQ ID NO: 11 - protein, 120 aa, synthetic construct
EVQLVQSGAEVKKPGESLKISCKGSGDSFISHWIAWVRQMPGKGLEWMGIVYPGDSDTRYSPSFQGQVTISADKSITTAYLQWSSLKASDTAMYYCARHEWELLGPFDYWGQGTLVTVSS

SEQ ID NO: 12 - protein, 121 aa, synthetic construct
EVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 13 - protein, 127 aa, synthetic construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 14 - protein, 119 aa, synthetic construct
QVQLVQSGAEVKKPGSSVKVSCKVSGGTFRSYAISWVRQAPGQGLEWMGGIIPIFDTRNYAQILQGRVTITADLSTSTAYMELNSLRSEDTAIYYCARGSDEGDWFDPWGQGTLVTVSS

SEQ ID NO: 15 - protein, 121 aa, synthetic construct
EVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSS

SEQ ID NO: 16 - protein, 30 aa, synthetic construct
QVQLVQSGAEVKKPGASVKVSCKASGYTFT

SEQ ID NO: 17 - protein, 5 aa, synthetic construct
SYGIS

SEQ ID NO: 18 - protein, 14 aa, synthetic construct
WVRQAPGQGLEWMG

SEQ ID NO: 19 - protein, 17 aa, synthetic construct
WISAYNGNTNYAQKLQG

SEQ ID NO: 20 - protein, 32 aa, synthetic construct
RVTMTTDTSTSTAYMELRSLRSDDTAVYYCAK

SEQ ID NO: 21 - protein, 13 aa, synthetic construct
DRHWHWWLDAFDY

SEQ ID NO: 22 - protein, 11 aa, synthetic construct
WGQGTLVTVSS

SEQ ID NO: 23 - protein, 30 aa, synthetic construct
QVQLVQSGSELKKPGASVKISCKASGYDFT

SEQ ID NO: 24 - protein, 5 aa, synthetic construct
NYAMN

SEQ ID NO: 25 - protein, 14 aa, synthetic construct
WVRQAPGHGLEWMG

SEQ ID NO: 26 - protein, 17 aa, synthetic construct
WINANTGDPTYAQGFTG

SEQ ID NO: 27 - protein, 32 aa, synthetic construct
RFVFSLDTSVSTAYLQISSLKAEDSAVYYCTR

SEQ ID NO: 28 - protein, 11 aa, synthetic construct
ERFLEWLHFDY

SEQ ID NO: 29 - protein, 11 aa, synthetic construct
WGQGTLVTVSS

SEQ ID NO: 30 - protein, 30 aa, synthetic construct
QVQLVQSGAEVKKPGASVKVSCKVSGYTLT

SEQ ID NO: 31 - protein, 5 aa, synthetic construct
ELSMH

SEQ ID NO: 32 - protein, 14 aa, synthetic construct
WVRQAPGKGLEWMG

SEQ ID NO: 33 - protein, 17 aa, synthetic construct
GFDPEYGKTFFAQNFQG

SEQ ID NO: 34 - protein, 32 aa, synthetic construct
RVTMTEDTSADTAYMELSSLRSEDTAVYYCAT

SEQ ID NO: 35 - protein, 16 aa, synthetic construct
EGYYETTTYYYNLFDS

SEQ ID NO: 36 - protein, 11 aa, synthetic construct
WGQGTLVTVSS

SEQ ID NO: 37 - protein, 30 aa, synthetic construct
QVQLVQSGSELKKPGASVKVSCKTSGYTFT

SEQ ID NO: 38 - protein, 5 aa, synthetic construct
DYAMT

SEQ ID NO: 39 - protein, 14 aa, synthetic construct
WVRQAPGQGLEWMG

SEQ ID NO: 40 - protein, 17 aa, synthetic construct
WITTNTGDPTYAPGFTG

SEQ ID NO: 41 - protein, 32 aa, synthetic construct
RFVFSLDTSVSTAYLQISSLKAEDTAVYYCAR

SEQ ID NO: 42 - protein, 10 aa, synthetic construct
VYHWIRGFEF

SEQ ID NO: 43 - protein, 11 aa, synthetic construct
WGQGTLVTVSS

SEQ ID NO: 44 - protein, 30 aa, synthetic construct
QVQLQESGPGLVKPSETLSLTCTVSGGSFS

SEQ ID NO: 45 - protein, 7 aa, synthetic construct
SSSSYWG

SEQ ID NO: 46 - protein, 14 aa, synthetic construct
WIRQPPGKGLEWIG

SEQ ID NO: 47 - protein, 16 aa, synthetic construct
SFYYSGNTYYNPSLKS

SEQ ID NO: 48 - protein, 32 aa, synthetic construct
RVTISEDTSKNQFSLKLSSVTAADTAVYYCAR

SEQ ID NO: 49 - protein, 16 aa, synthetic construct
QTYSSSWDGVLYYFDY

SEQ ID NO: 50 - protein, 11 aa, synthetic construct
WGQGTLVTVSS

SEQ ID NO: 51 - protein, 30 aa, synthetic construct
QVQLQESGPGLVKPSETLSLTCTVSNGSIS

SEQ ID NO: 52 - protein, 5 aa, synthetic construct
TYYWS

SEQ ID NO: 53 - protein, 14 aa, synthetic construct
WIRQPPGKGLEWIG

SEQ ID NO: 54 - protein, 16 aa, synthetic construct
YVYYTGRTKYNPSLKS

SEQ ID NO: 55 - protein, 32 aa, synthetic construct
RVTISVDTSKNQFSLNLSSVTAADTAVYYCAR

SEQ ID NO: 56 - protein, 18 aa, synthetic construct
GGIVVVPAARDYYYYMDV

SEQ ID NO: 57 - protein, 11 aa, synthetic construct
WGKGTTVTVSS

SEQ ID NO: 58 - protein, 30 aa, synthetic construct
EVQLVQSGAEVKKPGESLKIACKGSGFSFT

SEQ ID NO: 59 - protein, 5 aa, synthetic construct
SHWIG

SEQ ID NO: 60 - protein, 14 aa, synthetic construct
WVRQKPGRGLEWMG

SEQ ID NO: 61 - protein, 17 aa, synthetic construct
VIYPGDSDTRYSPSFQG

SEQ ID NO: 62 - protein, 32 aa, synthetic construct
QVTVSADKSINTAYLQWNSLKASDTAIYYCAR

SEQ ID NO: 63 - protein, 11 aa, synthetic construct
PNSGSPRYFEF

SEQ ID NO: 64 - protein, 11 aa, synthetic construct
WGRGTLVTVSS

SEQ ID NO: 65 - protein, 30 aa, synthetic construct
QVQLQESGPGLVKPSETLSLTCTVSGGSIS

SEQ ID NO: 66 - protein, 7 aa, synthetic construct
SSSYYWG

SEQ ID NO: 67 - protein, 14 aa, synthetic construct
WIRQPPGKGLEWIG

SEQ ID NO: 68 - protein, 16 aa, synthetic construct
SFYYSGNTYYNPSLKS

SEQ ID NO: 69 - protein, 32 aa, synthetic construct
RVTISVDTSKNQFSLKLSSVTAADTAVYYCAR

SEQ ID NO: 70 - protein, 16 aa, synthetic construct
QEYYYGSGSPSYYFDY

SEQ ID NO: 71 - protein, 11 aa, synthetic construct
WGQGTLVTVSS

SEQ ID NO: 72 - protein, 30 aa, synthetic construct
EVQLVQSGAEVKKPGESLKISCKGSGDSFI

SEQ ID NO: 73 - protein, 5 aa, synthetic construct
SHWIA

SEQ ID NO: 74 - protein, 14 aa, synthetic construct
WVRQMPGKGLEWMG

SEQ ID NO: 75 - protein, 17 aa, synthetic construct
IVYPGDSDTRYSPSFQG

SEQ ID NO: 76 - protein, 32 aa, synthetic construct
QVTISADKSITTAYLQWSSLKASDTAMYYCAR

SEQ ID NO: 77 - protein, 11 aa, synthetic construct
HEWELLGPFDY

SEQ ID NO: 78 - protein, 11 aa, synthetic construct
WGQGTLVTVSS

SEQ ID NO: 79 - protein, 30 aa, synthetic construct
EVQLVQSGAEVKKPGSSVKVSCKASGGTST

SEQ ID NO: 80 - protein, 5 aa, synthetic construct
NDAIS

SEQ ID NO: 81 - protein, 14 aa, synthetic construct
WVRQTPGQGLEWMG

SEQ ID NO: 82 - protein, 17 aa, synthetic construct
SIIPILDTTDHAQKFQG

SEQ ID NO: 83 - protein, 32 aa, synthetic construct
RVTITADKSTNTAYMELNSLRSDDTAVYYCAR

SEQ ID NO: 84 - protein, 12 aa, synthetic construct
EHIAARQDYFDY

SEQ ID NO: 85 - protein, 11 aa, synthetic construct
WGQGTLVTVSS

SEQ ID NO: 86 - protein, 30 aa, synthetic construct
EVQLVQSGSKLKKPGASVKVSCKASGYTFT

SEQ ID NO: 87 - protein, 5 aa, synthetic construct
SYTMN

SEQ ID NO: 88 - protein, 14 aa, synthetic construct
WVRQAPGQGLEWMG

SEQ ID NO: 89 - protein, 17 aa, synthetic construct
WINTDTGDPTYAQGFTG

SEQ ID NO: 90 - protein, 32 aa, synthetic construct
RFVFSLDTSVSTAFLQINSLKAEDTAVYYCAR

SEQ ID NO: 91 - protein, 18 aa, synthetic construct
GDCDSTSCYRYSYGYEDY

SEQ ID NO: 92 - protein, 11 aa, synthetic construct
WGQGTLVTVSS

SEQ ID NO: 93 - protein, 30 aa, synthetic construct
QVQLVQSGAEVKKPGSSVKVSCKVSGGTFR

SEQ ID NO: 94 - protein, 5 aa, synthetic construct
SYAIS

SEQ ID NO: 95 - protein, 14 aa, synthetic construct
WVRQAPGQGLEWMG

SEQ ID NO: 96 - protein, 17 aa, synthetic construct
GIIPIFDTRNYAQILQG

SEQ ID NO: 97 - protein, 32 aa, synthetic construct
RVTITADLSTSTAYMELNSLRSEDTAIYYCAR

SEQ ID NO: 98 - protein, 10 aa, synthetic construct
GSDEGDWFDP

SEQ ID NO: 99 - protein, 11 aa, synthetic construct
WGQGTLVTVSS

SEQ ID NO: 100 - protein, 30 aa, synthetic construct
EVQLVQSGTEVRKPGSSVKVSCKASGGTFS

SEQ ID NO: 101 - protein, 5 aa, synthetic construct
NYAIS

SEQ ID NO: 102 - protein, 14 aa, synthetic construct
WVRQAPGQGLEWMG

SEQ ID NO: 103 - protein, 17 aa, synthetic construct
SIIPILGTTDHAQKFQD

SEQ ID NO: 104 - protein, 32 aa, synthetic construct
RVTITADKSSNTTYMELSSLRSDDTAVYYCAR

SEQ ID NO: 105 - protein, 12 aa, synthetic construct
EYIAARLDYFDS

SEQ ID NO: 106 - protein, 11 aa, synthetic construct
WGQGTLVTVSS

SEQ ID NO: 107 - DNA, 381 bp, synthetic construct
  source 1..381: mol_type = other DNA
gaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagt

SEQ ID NO: 108 - DNA, 366 bp, synthetic construct
  source 1..366: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacgcctttgattattggggccaaggtaccctggtcaccgtctccagt

SEQ ID NO: 109 - DNA, 360 bp, synthetic construct
  source 1..360: mol_type = other DNA
caggtgcagctggtgcagtctgggtctgagttgaagaagcctggggcctcagtgaagatttcctgcaaggcttctggatacgacttcactaactatgctatgaattgggtgcgacaggcccctggacacgggcttgagtggatgggatggatcaacgccaacactggggacccaacgtatgcccagggcttcacaggacggtttgtcttctccttggacacctctgtcagcacggcatatctgcagatcagcagtttaaaggctgaggactctgccgtgtattactgtacgagagagcgatttttggagtggttacactttgactactggggccagggaaccctggtcaccgtctccagt

SEQ ID NO: 110 - DNA, 375 bp, synthetic construct
  source 1..375: mol_type = other DNA
caggtgcagctggtgcagtctggggctgaggtgaagaagcctggggcctcagtgaaggtctcctgcaaggtttccggatacaccctcactgaattatccatgcactgggtgcgacaggctcctggtaaagggcttgaatggatgggaggctttgatcctgagtatggtaaaacattcttcgcacagaacttccagggcagagtcaccatgaccgaggacacatctgcagacacagcctacatggagctaagcagcctgagatctgaggacacggccgtgtattactgtgcaacagaggggtattatgagactactacttattactacaacctttttgactcctggggccagggaaccctggtcaccgtctccagt

SEQ ID NO: 111 - DNA, 357 bp, synthetic construct
  source 1..357: mol_type = other DNA
caggtgcagctggtgcaatctgggtctgaattgaagaagcctggggcctcagtgaaggtttcctgcaagacttctggatacaccttcactgactatgctatgacttgggtgcgacaggcccctggacaagggcttgaatggatgggatggatcaccaccaacactggggacccaacgtatgccccgggcttcacaggacggtttgtcttctccttggacacctctgtcagcacggcatatctgcagatcagcagcctaaaggccgaggacactgccgtatattactgtgcgagagtgtatcattggatacggggatttgagttttggggccagggaaccctggtcaccgtctccagt

SEQ ID NO: 112 - DNA, 378 bp, synthetic construct
  source 1..378: mol_type = other DNA
caggtgcagctgcaggagtcgggcccaggactggtgaagccttcggagaccctgtccctcacctgcactgtctctggtggctccttcagcagtagtagttcctactggggctggatccgccagcccccagggaaggggctggagtggattgggagtttctattatagtgggaacacctactacaacccgtccctcaagagtcgagtcaccatatccgaagacacgtccaagaaccagttctccctgaagctgagctctgtgaccgccgcagacacggctgtgtattactgtgcgagacagacgtatagcagcagctgggacggggtcctgtactactttgactactggggccagggaaccctggtcaccgtctccagt

SEQ ID NO: 113 - DNA, 378 bp, synthetic construct
  source 1..378: mol_type = other DNA
caggtgcagctgcaggagtcggggccaggactggtgaagccttcggagaccctgtccctcacctgcactgtctctaatggctccatcagtacttactactggagctggatccggcagcccccagggaaggggctggagtggattggatatgtctattacactgggcgcaccaagtacaacccctccctcaagagtcgagtcaccatatcagtagacacgtccaagaaccagttctccctgaacctgagttctgtgaccgctgcggacacggccgtgtattactgtgcgagagggggtattgtagtagtcccagctgcgcgggactattactactacatggacgtctggggcaaagggaccacggtcaccgtctccagt

SEQ ID NO: 114 - DNA, 360 bp, synthetic construct
  source 1..360: mol_type = other DNA
gaggtgcaactggtgcagtctggagcagaggtgaaaaagcccggggagtctctgaagatcgcctgtaagggttctggattcagttttaccagccactggatcggctgggtgcgccagaagcccgggagaggcctggagtggatgggggtcatctatcctggtgactctgataccagatacagcccgtccttccaaggccaggtcaccgtctcagccgacaagtccatcaataccgcctacctgcagtggaacagcctgaaggcctcggacaccgccatatattactgtgcgagaccgaacagtgggagtccccggtacttcgagttctggggccgtggcaccctggtcaccgtctccagt

SEQ ID NO: 115 - DNA, 378 bp, synthetic construct
  source 1..378: mol_type = other DNA
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtttctattatagtgggaacacctactacaacccgtccctcaagagtcgagtcaccatatccgtagacacgtccaagaaccagttctccctgaagctgagctctgtgaccgccgcagacacggctgtgtattactgtgcgagacaggagtattactatggttcggggagtccttcgtactactttgactactggggccagggaaccctggtcaccgtctccagt

SEQ ID NO: 116 - DNA, 360 bp, synthetic construct
  source 1..360: mol_type = other DNA
gaggtgcagctggtgcagtctggagcagaggtgaaaaagcccggggagtctctgaagatctcctgtaagggttctggagacagttttatcagccactggatcgcctgggtgcgccagatgcccgggaaaggcctggagtggatggggatcgtctatcctggtgactctgataccagatacagcccgtccttccaaggccaggtcaccatctcagccgacaagtccatcaccaccgcctacttgcagtggagcagcctgaaggcctcggacaccgccatgtattactgtgcgagacacgagtgggaactacttggcccctttgactactggggccagggaaccctggtcaccgtctccagt

SEQ ID NO: 117 - DNA, 363 bp, synthetic construct
  source 1..363: mol_type = other DNA
gaggtgcagctggtgcagtctggggctgaggtgaagaagcctgggtcctcggtgaaggtctcctgcaaggcttctggaggcacctccactaacgatgctatcagttgggtgcgacagacccctggacaagggcttgagtggatgggaagtatcatccctatccttgatacaacagaccacgcacagaagttccagggcagagtcacgattaccgcggacaaatccacgaacacagcctacatggagctgaacagcctgagatctgatgacacggccgtgtattactgtgcgagagagcatatagcagctcgtcaggactactttgactattggggccagggaaccctggtcaccgtctccagt

SEQ ID NO: 118 - DNA, 381 bp, synthetic construct
  source 1..381: mol_type = other DNA
gaggtgcagctggtgcagtctgggtctaaattgaagaagcctggggcctcagtgaaggtttcctgcaaggcttctggatacaccttcactagctatactatgaattgggtgcgacaggcccctggacaagggcttgagtggatgggatggatcaacaccgacactggggacccaacgtatgcccagggcttcacaggacggtttgtcttctccttggacacctctgtcagcacggcatttctacagatcaacagcctaaaggctgaggacactgccgtatattactgtgcgagaggagattgtgatagtaccagctgctatagatacagttatggttacgaggactactggggccagggaaccctggtcaccgtctccagt

SEQ ID NO: 119 - DNA, 357 bp, synthetic construct
  source 1..357: mol_type = other DNA
caggtgcagctggtgcagtctggggctgaggtgaagaagcctgggtcctcggtgaaggtctcctgcaaggtttctggaggcaccttcaggagctatgctatcagctgggtgcgacaggcccctggacaagggcttgagtggatgggagggatcatccctatctttgatacaagaaactacgcacagattcttcagggcagagtcacgattaccgcggacttatccacgagcacagcctacatggagctgaacagtctgagatctgaggacacggccatttattactgtgcgagagggagcgacgagggggactggttcgacccctggggccaaggaaccctggtcaccgtctccagt

SEQ ID NO: 120 - DNA, 363 bp, synthetic construct
  source 1..363: mol_type = other DNA
gaggtgcagctggtgcagtctgggactgaggtgaggaagcctgggtcctcggtgaaggtctcctgcaaggcttctggaggcaccttcagcaactatgctatcagctgggtgcgacaggcccctggacaggggcttgagtggatgggaagtatcatccctatccttggaacaacagaccacgcacagaagttccaggacagagtcacgattaccgcggacaaatcctcgaacacaacctacatggagctgagcagcctgagatctgatgacacggccgtatattactgtgcgagagagtatatagcagctcgtctggactactttgactcttggggccagggaaccctggtcaccgtctccagt

SEQ ID NO: 121 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 123 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 124 - protein, 107 aa, synthetic construct [sequence omitted]

SEQ ID NO: 125 - DNA, 320 bp, synthetic construct
  source 1..320: mol_type = other DNA
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 126 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 127 - protein, 6 aa, synthetic construct
QSISSY

SEQ ID NO: 128 - skipped

SEQ ID NO: 129 - protein, 9 aa, synthetic construct
QQSYSTPPT

SEQ ID NO: 130 - DNA, 294 bp, synthetic construct
  source 1..294: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 131 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 132 - DNA, 45 bp, synthetic construct
  source 1..45: mol_type = other DNA
gagcccaaatcttgtgacaaaactcacacatgcccaccgtgccca

SEQ ID NO: 133 - protein, 15 aa, synthetic construct
EPKSCDKTHTCPPCP

SEQ ID NO: 134 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 135 - DNA, 330 bp, synthetic construct
  source 1..330: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 136 - protein, 106 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - DNA, 321 bp, synthetic construct
  source 1..321: mol_type = other DNA
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 138 - protein, 106 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 139 - DNA, 321 bp, synthetic construct
  source 1..321: mol_type = other DNA
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 140 - protein, 6 aa, synthetic construct
AASLQS

(19) United States
(12) Patent Application Publication
(10) Pub. No.: US 2024/0425604 A1
(43) Pub. Date: Dec. 26, 2024

(54) TREATMENT OF IMMUNE CHECKPOINT INHIBITOR-TREATED CANCERS WITH HIGH EGFR EXPRESSION USING AN ANTIBODY THAT BINDS AT LEAST EGFR

(71) Applicant: MERUS N.V., Utrecht (NL)

(72) Inventors: Ernesto Isaac WASSERMAN, Utrecht (NL); Jeroen Jilles LAMMERTS VAN BUEREN, Utrecht (NL)

(21) Appl. No.: 18/694,252

(22) PCT Filed: Oct. 6, 2022
(86) PCT No.: PCT/NL2022/050563

§ 371 Date: Mar. 21, 2024

(30) Foreign Application Priority Data
Oct. 6, 2021 (NL) 2029327

(51) Int. Cl. C07K 16/28; A61K 39/00; A61P 35/00

(52) U.S. Cl. CPC C07K 16/2863; A61P 35/00; A61K 2039/54; A61K 2039/545; C07K 2317/31

(57) ABSTRACT

The disclosure relates to means and methods in the treatment of cancer. The disclosure in particular relates to a method of treating a cancer in an individual with an antibody that at least binds EGFR. The invention further relates to the use in such methods and to use in the manufacture of a medicament for the treatment of a cancer having particular EGFR levels. Such antibodies are particularly useful in the treatment of cancers such as gastric, esophageal, gastroesophageal-junction or head and neck cancer.

### CROSS REFERENCE TO RELATED APPLICATIONS

This application is a 35 U.S.C. § 371 National Phase Application of PCT/NL2022/050563, filed Oct. 6, 2022, which claims priority to NL Application No. 2029327A, filed Oct. 6, 2021.

### REFERENCE TO A SEQUENCE LISTING SUBMITTED ELECTRONICALLY VIA EFS-WEB

This application includes a Sequence Listing submitted electronically via EFS-Web (Name: “4096_0570001_SequenceListing_ST26.xml”; Size: 133,967 Bytes; Date of Creation: Mar. 18, 2024), which is hereby incorporated by reference in its entirety.

### FIELD OF THE INVENTION

The disclosure relates to means and methods in the treatment of cancer. The disclosure in particular relates to a method of treating a cancer in an individual with an antibody that at least binds EGFR. The invention further relates to the use in such methods and to use in the manufacture of a medicament for the treatment of a cancer having particular EGFR levels. Such antibodies are particularly useful in the treatment of cancers such as gastric, esophageal, gastro-esophageal-junction or head and neck cancer.

### BACKGROUND OF THE INVENTION

Traditionally, most cancer drug discovery has focused on agents that block essential cell functions and kill dividing cells via chemotherapy. However, chemotherapy rarely results in a complete cure. In most cases, the tumors in the patients stop growing or temporarily shrink only to start proliferating again, some times more rapidly, and become increasingly more difficult to treat.

Cancer is still a major cause of death in the world, in spite of the many advances that have been made in the treatment of the disease and the increased knowledge of the molecular events that lead to cancer.

It has been reported that, in the United States, head and neck cancer, in particular in the oral cavity and pharynx, already accounts for 3 percent of malignancies, with approximately 53,000 Americans developing such cancer annually and 10,800 dying therefrom (Siegel et al., CA Cancer J Clin. 2020;70(1): 7. Epub 2020 Jan. 8.). Furthermore, head and neck squamous cell carcinoma (HNSCC) is reported to be the sixth leading cancer by incidence worldwide, with a five-year overall survival rate of patients with HNSCC of about 40-50% (in Head and Neck Cancer, Union for International Cancer Control, 2014 Review of Cancer Medicines on the WHO List of Essential Medicines). A meta-analysis into locoregionally advanced head and neck squamous cell carcinoma (LA-HNSCC) reported that the addition of an anti-EGFR agent to radiotherapy or chemoradiotherapy did not improve clinical outcomes in patients with LA-HNSCC (Oncotarget. 2017; 8(60): 102371-102380). Also, the addition of anti-EGFR agents was reported to increase the risk of skin toxicities and mucositis.

Furthermore, gastric cancer, is the 5^{th} most common diagnosed cancer worldwide and the 3^{rd} mostly deadly. In 2018, an estimated 783,000 deaths were due to gastric cancer. Esophageal cancer is the 9^{th} most common cancer and the 6^{th} most common cause of cancer deaths. It has been reported that epidermal growth factor receptor (EGFR) is overexpressed in more than 30% of gastric adenocarcinoma (GAC) and esophageal adenocarcinoma (EAC) cases. However, an analysis reviewing of six different studies concluded that the addition of an anti-EGFR agent to chemotherapy did not meaningfully improve overall survival or progression free survival for patients with advanced/metastatic EAC, GAC or Gastro-esophageal junction adenocarcinoma (GEJAC) (Kim et al. 2017 Oncotarget. 2017 Nov. 17; 8(58): 99033-99040).

A need thus exists for improved cancer treatments, in particular for treatment of gastric, esophageal and head and neck cancer.

### SUMMARY OF THE INVENTION

The disclosure provides the following preferred aspects. However, the invention is not limited thereto.

The present disclosure provides an antibody or functional part, derivative and/or analogue thereof that comprises a first variable domain that binds an extracellular part of EGFR for use in the treatment of a cancer in a subject and which cancer expresses EGFR or EGFR and LGR5.

The present disclosure provides an antibody or functional part, derivative and/or analogue thereof that comprises a first variable domain that binds an extracellular part of EGFR for use in the treatment of a cancer in a subject, which cancer in said subject has progressed after having received prior treatment with an immune checkpoint inhibitor and which cancer expresses EGFR or EGFR and LGR5.

The present disclosure also provides the use of an antibody or functional part, derivative and/or analogue thereof that comprises a variable domain that binds an extracellular part of EGFR in the manufacture of a medicament for treating a cancer in a subject, which cancer in said subject has progressed after having received prior treatment with an immune checkpoint inhibitor and which cancer expresses EGFR or EGFR and LGR5.

The present disclosure also provides a method of treating a subject having an EGFR expressing cancer, wherein said subject has progressed after having received prior treatment with an immune checkpoint inhibitor, the method comprising providing the subject with an effective amount of an antibody or functional part, derivative and/or analogue thereof that comprises a first variable domain that binds an extracellular part of EGFR.

In certain aspects, the cancer of the present disclosure is in particular gastric cancer, esophageal cancer, gastric-esophageal-junction cancer or head and neck cancer. The head and neck cancer in particular is head and neck squamous cell carcinoma (HNSCC). The gastric cancer, esophageal cancer, gastric-esophageal-junction cancer in particular is an adenocarcinoma. Said esophageal cancer can also be a squamous cell carcinoma.

In certain aspects, the cancer of the present disclosure is in particular gastric, esophageal or gastric-esophageal-junction cancer having an EGFR expression characterized by an IHC score of 3+. In certain aspects, the cancer of the present disclosure is gastric, esophageal or gastric-esophageal-junction cancer having an EGFR expression characterized by an H score for EGFR of more than 200.

The present disclosure also provides an antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an IHC score of 3+. The present disclosure also provides an antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an H score for EGFR of more than 200.

The present disclosure also provides an antibody or functional part, derivative and/or analogue thereof that comprises a first variable domain that binds an extracellular part of EGFR for use in the treatment of head and neck cancer, gastric, esophageal or gastric-esophageal-junction cancer in a subject, wherein said cancer is characterized by comprising an EGFR gene amplification. Such gene amplification of EGFR is in certain aspects characterized by an EGFR copy number of 8 or more, or a level of circulating tumor DNA (ctDNA) of at least 2.14, or at least 2.5.

In certain aspects, EGFR mRNA amplification is defined as eligible by an EGFR copy number of 8 or more (such as defined by next-generation sequencing), or ctDNA of at least 2.14 or at least 2.5.

In some aspects, the subject has progressed after having received prior treatment with an immune checkpoint inhibitor. In certain aspects, said immune checkpoint inhibitor comprises a PD-L1, PD-1, CTLA-4, B7-1 or B7-2 inhibitor. In certain aspects, such inhibitors comprise antibodies targeting PD-L1, PD-1, CTLA-4, B7-1 or B7-2 inhibitor. In certain aspects, the immune checkpoint inhibitors comprise durvalumab, retifanlimab, cemiplimab, pembrolizumab, ipilimumab, nivolumab or atezolizumab.

In certain aspects, the subject of the present disclosure has not received prior treatment with an anti-EGFR agent. In certain aspects, the subject has not received prior treatment with an antibody targeting EGFR, or the subject has not received prior treatment with cetuximab.

In certain aspects, the gastric, esophageal or gastric-esophageal-junction cancer of the present disclosure expresses EGFR characterized by an H score of between more than 200 but not more than 300. In certain aspects, said H score for EGFR is determined using immunohistochemistry (IHC).

In certain aspects, the subject of the present disclosure is a mammalian subject, such as a human subject.

In certain aspects, the treatment of the present disclosure comprises providing the subject with an effective amount of said antibody or functional part, derivative and/or analogue thereof. In certain aspects, said treatment comprises providing a flat dose of between 500 mg to 2000 mg. In certain aspects, said dose is between 1100 mg to 1800 mg. In certain aspects, said dose is between 1100 mg to 1500 mg. In certain aspects, said treatment comprises a flat dose of 1500 mg of the antibody or functional part, derivative and/or analogue thereof to the subject. In certain aspects, the antibody or functional part, derivative and/or analogue thereof is provided intravenously to the subject. In certain aspects, the antibody or functional part, derivative and/or analogue thereof is provided weekly, biweekly or monthly. In certain aspects, the antibody or functional part, derivative and/or analogue thereof is provided biweekly.

In certain aspects, the antibody or functional part, derivative and/or analogue thereof is ADCC enhanced. Also, in certain aspects, the antibody or functional part, derivative and/or analogue thereof is afucosylated.

In certain aspects, the antibody or functional part, derivative and/or analogue thereof of the present disclosure is a multispecific antibody. In certain aspects, the antibody or functional part, derivative and/or analogue thereof of the present disclosure is a bispecific antibody that at least binds EGFR. In certain aspects, the antibody comprises a second variable domain that does not bind EGFR. In certain aspects, the antibody comprises a second variable domain that binds LGR5.

The antibody or functional part, derivative and/or analogue thereof that comprises a first variable domain that binds an extracellular part of EGFR of the present disclosure is also referred herein to as a therapeutic agent.

### BRIEF DESCRIPTION OF THE DRAWINGS

FIG. 1 Human LGR5 sequence; Sequence ID NO: 1.

FIG. 2 Human EGFR sequence; Sequence ID NO: 2.

FIG. 3 (a) Amino acid sequences of heavy chain variable regions (Sequence ID Nos: 3-15) that together with a common light chain variable region such as the variable region of the human kappa light chain IgVK1 39*01/IGJK1*01 form a variable domain that binds LGR5 and EGFR. The CDR and framework regions are indicated in FIG. 3b. Respective DNA sequence are indicated in FIG. 3c.

FIG. 4 a) Amino acid sequence of a common light chain amino acid sequence. b) Common light chain variable region DNA sequence and translation (IGKV1-39/jk1). c) Light chain constant region DNA sequence and translation. d) V-region IGKV1-39A; e) CDR1, CDR2 and CDR3 of a common light chain according to IMGT numbering.

FIG. 5 IgG heavy chains for the generation of bispecific molecules. a) CH1 region DNA sequence and translation. b) Hinge region DNA sequence and translation. c) CH2 region DNA sequence and translation. d) CH3 domain containing variations L351K and T366K (KK) DNA sequence and translation. e) CH3 domain containing variations L351D and L368E (DE) DNA sequence and translation. Residue positions are according to EU numbering.

### DETAILED DESCRIPTION OF THE DISCLOSURE

In order that the present description may be more readily understood, certain terms are first defined. Additional definitions may be set forth throughout the detailed description where deemed required. Unless separately defined herein, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents. Use of the term “comprising” “having” “including” as well as other forms, such as “comprise”, “comprises”, “comprised”, “has”, “have”, “had”, “include”, “includes”, and “included”, is not limiting.

The term “antibody” as used herein means a proteinaccous molecule belonging to the immunoglobulin class of proteins, containing one or more domains that bind an epitope on an antigen, where such domains are or derived from or share sequence homology with the variable region of an antibody. Antibodies are typically made of basic structural units, each with two heavy chains and two light chains. An antibody according to the present invention is not limited to any particular format or method of producing it.

A “bispecific antibody” is an antibody as described herein wherein one domain of the antibody binds to a first antigen whereas a second domain of the antibody binds to a second antigen, wherein said first and second antigens are not identical, or where one domain binds a first epitope on an antigen, whereas a second domain binds to a second epitope on the antigen. The term “bispecific antibody” also encompasses antibodies wherein one heavy chain variable region/light chain variable region (VH/VL) combination binds a first antigen or epitope on an antigen and a second VH/VL combination that binds a second antigen or epitope on the antigen. The term further includes antibodies wherein VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full-length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. In certain aspects, the light chain of the present invention can also be a light chain as specified herein, having from 0 to 10 amino acid insertions, deletions, substitutions, additions or a combination thereof. In certain aspects, the light chain of the present invention can also be a light chain as specified herein, having from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The term ‘full length IgG’ or ‘full length antibody’ according to the invention is defined as comprising an essentially complete IgG, which however does not necessarily have all functions of an intact IgG. For the avoidance of doubt, a full-length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein variations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen binding specificity of the IgG. In certain aspects, such IgG molecules can have a deletion of between 1 and 10 amino acid residues in non-CDR regions, wherein the deleted amino acids are not essential for the antigen binding specificity of the IgG.

A “derivative of an antibody” is a protein that but for the CDR regions deviates from the amino acid sequence of a natural antibody in at most 20 amino acids. A derivative of an antibody as disclosed herein is an antibody that deviates from said amino acid sequence in at most 20 amino acids. The functional part, derivative and/or analogue maintains the binding specificity of the (bispecific) antibody. An “analogue of an antibody” is a protein that may be different in structure, format or origin but maintains the binding specificity of the antibody it is an analogue of.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Advance® 11.5.2 software using the default settings, which employ a modified ClustalW algorithm (Thompson, J. D., Higgins, D. G., and Gibson T. J., (1994) Nuc. Acid Res. 22 (22): 4673-4680), the swgapdnamt score matrix, a gap opening penalty of 15 and a gap extension penalty of 6.66. Amino acid sequences are aligned with the AlignX application of the Vector NTI Advance® 11.5.2 software using default settings, which employ a modified ClustalW algorithm (Thompson, J. D., Higgins, D. G., and Gibson T. J., (1994) Nuc. Acid Res. 22 (22): 4673-4680), the blosum62mt2 score matrix, a gap opening penalty of 10 and a gap extension penalty of 0.1.

As an antibody typically recognizes an epitope of an antigen, and such an epitope may be present in other compounds as well, antibodies according to the present invention that “specifically recognize” an antigen, for example, EGFR or LGR5, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In terms of tumor development, an effective amount is an amount sufficient to delay tumor development. In terms of tumor recurrence, an effective amount is an amount sufficient to prevent or delay tumor recurrence. An effective amount can be administered in one or more administrations. The effective amount of the agent or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one aspect, an “effective amount” is the amount of an antibody as disclosed herein as the therapeutic agent to affect a decrease in a cancer (for example a decrease in the number of cancer cells); slowing of progression of a cancer or prevent regrowth or recurrence of the cancer. As mentioned before herein, the antibody or functional part, derivative and/or analogue thereof that binds EGFR or binds EGFR and LGR5 of the present disclosure, is also referred herein to as a “therapeutic agent”. In certain aspects, the effective amount herein is a flat dose of 1500 mg administered on a biweekly basis to a subject having a cancer of the present disclosure.

The term “flat dose” herein refers to a dosing regimen wherein a subject is administered with a fixed amount of a therapeutic substance over multiple administrations, independent of body weight of the subject. Flat dosing is typically abbreviated with qnw, wherein n is an integer indicating the interval and w is week. For instance, a q2w flat dose administration regimen of 1500 mg antibody means a fixed amount of 1500 mg antibody is administered each 2 weeks. Herein, in certain aspects, the therapeutic substance is an antibody binding EGFR or EGFR and LGR5 that is administered with a q2w dosing regimen of 1500 mg. In certain aspects, the subject has been administered at least 3 q2w flat dosages of 1500 mg. In certain aspects, said administration is at least 4 dosages or more and may last until the patient shows sufficient clinical or radiological progression.

The flat dose may be premedicated, meaning medication is administered to the subject prior to being administered the antibody of the present invention. In certain aspects, the flat dose of 1500 mg antibody is premedicated with an antihistamine, pain reducing medication, fever reducing medication and/or anti-inflammatory medication.

The term “H-score”, sometimes referred to as “histo” score in the art, refers to a reproducible and standardized scoring methodology which can be used to semi-quantitatively calculate expression of a gene of interest in a tumor sample following a protocol based on methods of immunohistochemistry (IHC) or in situ hybridization techniques (ISH), all well-known with the skilled person and follow the ASCO Apr. 10, 2015 posting how to calculate H-scores. See also Hirsch FR, Varella-Garcia M, Bunn PA Jr, et al: Epidermal growth factor receptor in non-small-cell lung carcinomas: Correlation between gene copy number and protein expression and impact on prognosis. J Clin Oncol 21:3798-3807, 2003; and John T, Liu G, Tsao M-S: Overview of molecular testing in non-small-cell lung cancer: Mutational analysis, gene copy number, protein expression and other biomarkers of EGFR for the prediction of response to tyrosine kinase inhibitors. Oncogene 28: S14-S23, 2009. The relevant teachings of these references are herein incorporated by reference.

In the context of H scoring for EGFR, the term “determined using IHC” refers to a method that uses or comprises IHC as the basis for subsequently determining the H score, as opposed to methods alternative to IHC.

In some aspects, the present disclosure provides an antibody or functional part, derivative and/or analogue thereof that comprises a first variable domain that binds an extracellular part of EGFR for use in the treatment of a cancer in a subject, which cancer in said subject has progressed after having received prior treatment with an immune checkpoint inhibitor and which cancer expresses EGFR.

In some aspects, the cancer is selected from gastric cancer, esophageal cancer, gastric-esophageal-junction cancer or head and neck cancer, in particular squamous cell carcinoma of the head and neck (SCCHN).

In certain aspects, the cancer is gastric, esophageal or gastric-esophageal-junction cancer having an EGFR expression characterized by an IHC score of 3+. In certain aspects, said cancer has an H score for EGFR of more than 200. In certain aspects, said IHC is the tumor membrane score.

Also provided in the present disclosure is an antibody or functional part, derivative and/or analogue thereof that comprises a first variable domain that binds an extracellular part of EGFR for use in the treatment of a cancer in a subject, wherein said cancer expresses EGFR which is characterized by an IHC score of 3+and wherein said variable domain comprises the amino acids as disclosed further herein.

Also provided in the present disclosure is an antibody or functional part, derivative and/or analogue thereof that comprises a first variable domain that binds an extracellular part of EGFR for use in the treatment of a cancer in a subject, wherein said cancer expresses EGFR which is characterized by an H score for EGFR of more than 200 and wherein said variable domain comprises the amino acids as disclosed further herein.

Also provided in the present disclosure is the use of an antibody or functional part, derivative and/or analogue thereof that comprises a variable domain that binds an extracellular part of EGFR in the manufacture of a medicament for treating a cancer in a subject, which cancer in said subject has progressed after having received prior treatment with an immune checkpoint inhibitor and which cancer expresses EGFR.

Also provided in the present disclosure is a method of treating a subject having an EGFR expressing cancer, wherein said subject has progressed after having received prior treatment with an immune checkpoint inhibitor, the method comprising providing the subject with an effective amount of an antibody or functional part, derivative and/or analogue thereof that comprises a first variable domain that binds an extracellular part of EGFR.

Also provided in the present disclosure is an antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an IHC score of 3+.

Also provided in the present disclosure is an antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an H score for EGFR of more than 200.

The words cancer and tumor are used herein and typically both refer to cancer, unless otherwise specifically stated.

Epidermal growth factor (EGF) receptor (EGFR, ErbB1, or HER1) is a member of a family of four receptor tyrosine kinases (RTKs), named Her- or cErbB-1, -2, -3 and -4. EGFR is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. A major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is GenBank NM 005228.3. This accession number is primarily given to provide a further method of identification of EGFR protein as a target, the actual sequence of the EGFR protein bound by an antibody may vary, for instance because of a mutation in the encoding gene such as those occurring in some cancers or the like.

Where reference herein is made to EGFR, the reference refers to human EGFR unless otherwise stated. The variable domain antigen-binding site that binds EGFR, binds EGFR and a variety of variants thereof such as those expressed on some EGFR positive tumors.

The term “LGR” refers to the family of proteins known as Leucine-rich repeat-containing G-protein coupled receptors. Several members of the family are known to be involved in the WNT signaling pathway, of note LGR4; LGR5 and LGR6.

LGR5 is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

In particular, the cancer is gastric, esophageal, or gastro-esophageal-junction cancer. Gastric cancer (also referred to as stomach cancer) is a cancer that develops from the lining of the stomach and in particular the mucus-producing glandular cells found therein. Such a cancer is also referred to as adenocarcinoma, or in this case gastric adenocarcinoma as is develops from the lining of the stomach. In particular, the cancer is thus a gastric adenocarcinoma or cancer that develops from the lining of the stomach which is used interchangeably herein. Esophageal cancer is cancer that develops from the esophagus. The two main subtypes are ESCC (esophageal squamous-cell carcinoma) and EAC (esophageal adenocarcinoma). Gastro-esophageal-junction cancer (also known as gastro-esophageal junction adenocarcinoma) arises from the gastro-esophageal junction.

Cancers that are known collectively as head and neck cancers usually originate in the squamous cells that line the moist, mucosal surfaces inside the head and neck, such as inside the mouth, the nose, and the throat. These squamous cell cancers are often referred to as squamous cell carcinomas of the head and neck and said cancers are treated in certain aspects of the present disclosure. Although rare, head and neck cancers can also occur in the salivary glands. In particular, the head and neck cancer may occur in the oral cavity. This includes the lips, the front two-thirds of the tongue, the gums, the lining inside the cheeks and lips, the floor of the mouth under the tongue, the hard palate, and the small area of the gum behind the wisdom teeth.

Thus, in particular, the head and neck cancer is squamous cell carcinoma and includes nasopharyngeal cancer, laryngeal cancer, hypopharyngeal cancer, nasal cavity cancer, paranasal sinus cancer, oral cancer, oropharyngeal cancer or salivary gland cancer. More in particular, the present invention relates to treatment of a cancer comprising a squamous cell head and neck cancer, such as located in the oropharynx, hypopharynx, the larynx, the oral cavity or the tongue.

Also, the head and neck cancer is in particular a squamous cell carcinoma of unknown primary (also referred to in the art as a cancer of unknown primary or CUP).

In the present disclosure, the cancer expresses EGFR or EGFR and LGR5.

As used herein, a cancer expresses EGFR if the cancer comprises cells that express EGFR. A cell which expresses EGFR comprises detectable levels of RNA that codes for EGFR. In certain aspects, EGFR expression is determined by ISH.

In certain aspects, EGFR protein expression is detected by IHC. In certain aspects, EGFR expression is determined by IHC using a commercially available EGFR detection kit, such as the EGFR pharmDx™ kit for a Dako autostainer (Agilent), using the manufacturer's recommendations or the commercially available IHC EGFR detection kit based on EGFR clone 113 which binds the EGFR extracellular domain (Leica, https://shop.leicabiosystems.com/us/ihc-ish/ihc-primary-antibodies/pid-epidermal-growth-factor-receptor). Alternatively, EGFR expression is determined using the Novocastra™ Liquid Mouse Monoclonal Antibody Epidermal Growth Factor Receptor which is based on clone EGFR.113 (Product Code: NCL-L-EGFR, Epidermal Growth Factor Receptor-IHC Primary Antibodies by leicabiosystems.com).

Briefly, the commercially available EGFR pharmDx™ IHC kit system contains reagents required to complete an IHC staining procedure for routinely-fixed, paraffin-embedded specimens. Following incubation with the primary, non-Her2, Her3 and Her4 cross-reactive, monoclonal antibody (clone 2-18C9) to human EGFR protein, this kit employs a ready-to-use visualization reagent based on dextran technology. This reagent consists of both secondary goat anti-mouse antibody molecules and horseradish peroxidase molecules linked to a common dextran polymer backbone. The enzymatic conversion of the subsequently added chromogen results in formation of a visible reaction product at the antigen site. Results are routinely assessed using a light microscope. Control slides containing two formalin-fixed, paraffin-embedded human cell lines with staining intensity scores of 2+and 0 are provided for quality control of the kit reagent performance.

Staining intensity is established as follows: 3+ (strong staining): visible at low levels of magnification, x5 objective lens which could be confirmed at higher levels as required; 2+ (moderate staining): visible at intermediate levels of magnification, x10 or x20 objective lenses; 1+ (weak staining): only reliably confirmable at high magnification, x40 objective lens; 0 (no staining): no staining visible at high magnification.

In certain aspects, EGFR expression is determined using immunohistochemistry (IHC) and the cancer is IHC positive for EGFR. In certain aspects, the cancer is gastric cancer, esophageal cancer, gastric-esophageal-junction cancer characterized by an EGFR IHC score of 3+.

In certain aspects, EGFR expression is determined using immunohistochemistry (IHC) followed by assigning an H-score for EGFR using a range of 0-300. In certain aspects, the cancer of the present disclosure is gastric cancer, esophageal cancer, gastric-esophageal-junction cancer characterized by an H-score for EGFR of more than 200 on a scale of 0-300. In certain aspects, the EGFR H score is thus >200 up to and including 300. In certain aspects, the cancer of the present disclosure is characterized by an H-score for EGFR of more than 50 on a scale of 0-300. In certain aspects, the cancer of the present disclosure is head and neck cancer characterized by an H-score for EGFR of more than 50 on a scale of 0-300. In certain aspects, the cancer of the present disclosure is characterized by an H-score for EGFR of more than 80 on a scale of 0-300. In certain aspects, the cancer of the present disclosure is head and neck cancer characterized by an H-score for EGFR of more than 80 on a scale of 0-300.

In another aspect, the cancer is a head and neck cancer characterized by an EGFR IHC score of 2+or 3+.

Herein, determining the H-score to assign the EGFR expression status involves a first step of establishing intensity of membrane staining (resulting in a scoring of 0, 1+, 2+, or 3+) which is determined for each cell in a predefined field as described herein. Subsequently, the percentage of cells at each staining intensity level is calculated, and finally, an H-score is assigned using the following formula: [1×(% cells having 1+staining)+2×(% cells having 2+staining+3×(% cells having 3+staining)] resulting in an H-score for EGFR between 0-300. As a result, the H-score gives more relative weight to higher intensity or amount of staining in a given tumor sample.

In certain aspects, said cancer of the present disclosure is characterized by comprising an EGFR gene amplification. In certain aspects, said cancer is gastric cancer. In certain aspects, said cancer is gastroesophageal junction adenocarcinoma. Said gene amplification of EGFR is in certain aspects characterized by an EGFR copy number based on a solid tissue sample of 8 or more, or an EGFR amplification score (also known as copy number alteration (CNA)) based on circulating tumor DNA (ctDNA) of at least 2.14, or at least 2.5 but in certain aspects, not more than 5.

In certain aspects, EGFR amplification is defined as an EGFR copy number of 8 or more (in particular defined as 8 copies or more above ploidy based on solid tissue amplification). In certain aspects, EGFR copy number is established by next-generation sequencing on a formalin-fixed paraffin embedded (FFPE) tissue sample.

In certain aspects, EGFR gene copy number is established using next-generation sequencing (NGS). In certain aspects, said NGS is performed on a solid tissue sample or a liquid sample, such as blood or plasma. In certain aspects, EGFR amplification score is established by next-generation sequencing on ctDNA resulting in a score of at least 2.14 or at least 2.5. In certain aspects, said ctDNA score is not more than 5. Said copy number assessment may be based on blood-derived cfDNA. As an example, determining of EGFR copy number can be performed as mentioned by Kato et al. 2019 (Revisiting Epidermal Growth Factor Receptor (EGFR) Amplification as a Target for Anti-EGFR Therapy: Analysis of Cell-Free Circulating Tumor DNA in Patients With Advanced Malignancies. JCO Precis Oncol 3: PO.18.00180).

Herein, the term “ctDNA” (circulating tumor DNA) is used interchangeably with “cfDNA” (cell-free tumor DNA).

In certain aspects, EGFR gene copy number is established using FISH. In certain aspects, said cancer is characterized by an EGFR/CEP7 ratio of at least 2.0 or higher. Establishing the EGFR/CEP7 ratio is standard in the art but may for instance be established using a commercially available kit, or be performed as described by Maron, et al., 2018 (Targeted Therapies for Targeted Populations: Anti-EGFR Treatment for EGFR-Amplified Gastroesophageal Adenocarcinoma. Cancer Discov 8:696-713). The EGFR FISH test is designed to detect amplification of the EGFR locus (positioned at chromosome 7p11.2). In such a methodology, FISH is performed on sections of formalin-fixed, paraffin-embedded tumor tissue. Slides are prepared per standard protocols and 100 interphase cells are scored. Cutoff for amplification is next set at ≥2.0 ratio of EGFR: CEP7.

Optionally, the treatment with the antibody or functional part, derivative and/or analogue thereof comprises (or in certain aspects is preceded by) a step of diagnosing the subject for EGFR status. In certain aspects, subjects having gastric cancer, esophageal cancer, gastric-esophageal-junction cancer characterized by an IHC score of 3+, or said cancer is characterized by an H-score for EGFR of more than 200, on a scale of 0-300, are selected for treatment. In certain aspects, the treatment of a subject is preceded by a step of diagnosing said subject of having a gastric cancer, esophageal cancer, gastric-esophageal-junction cancer characterized by an H-score for EGFR of more than 200 on a scale of 0-300.

In certain aspects, subjects having a cancer, such as gastric, esophageal cancer, gastric-esophageal-junction cancer characterized by an EGFR gene amplification which comprises an EGFR/CEP7 ratio of at least 2.0 or higher; an EGFR copy number of 8 or more; or an EGFR ctDNA score of at least 2.14 or at least 2.5, are selected for treatment. In certain aspects, the treatment of a subject is preceded by a step of diagnosing said subject of having a gastric cancer, esophageal cancer, gastric-esophageal-junction cancer characterized by an EGFR gene amplification which comprises an EGFR/CEP7 ratio of at least 2.0 or higher; an EGFR copy number of 8 or more; or an EGFR ctDNA score of at least 2.14 or at least 2.5.

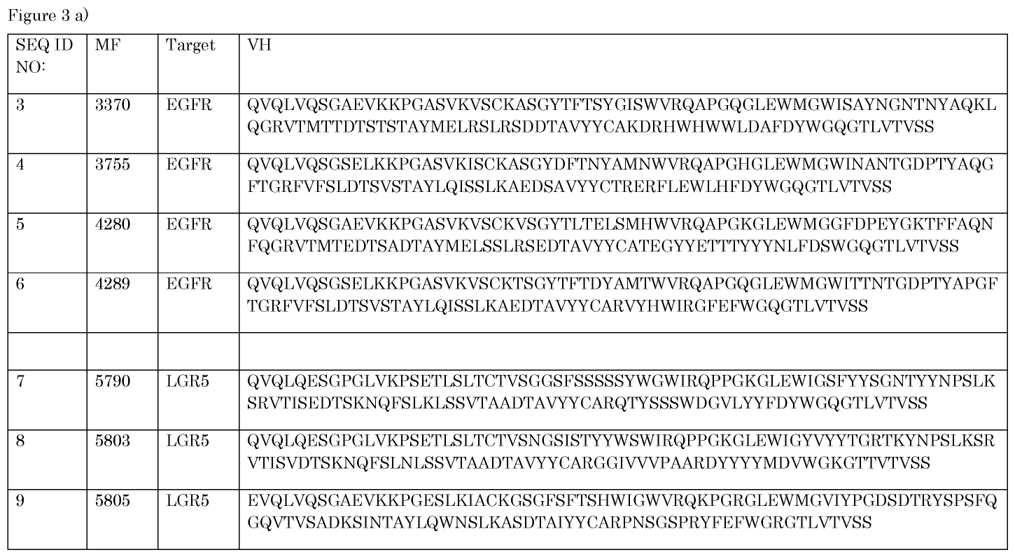

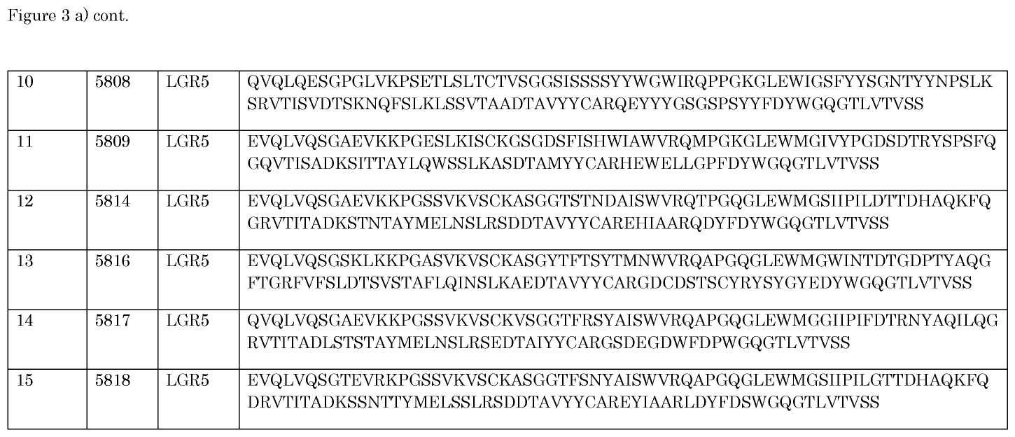

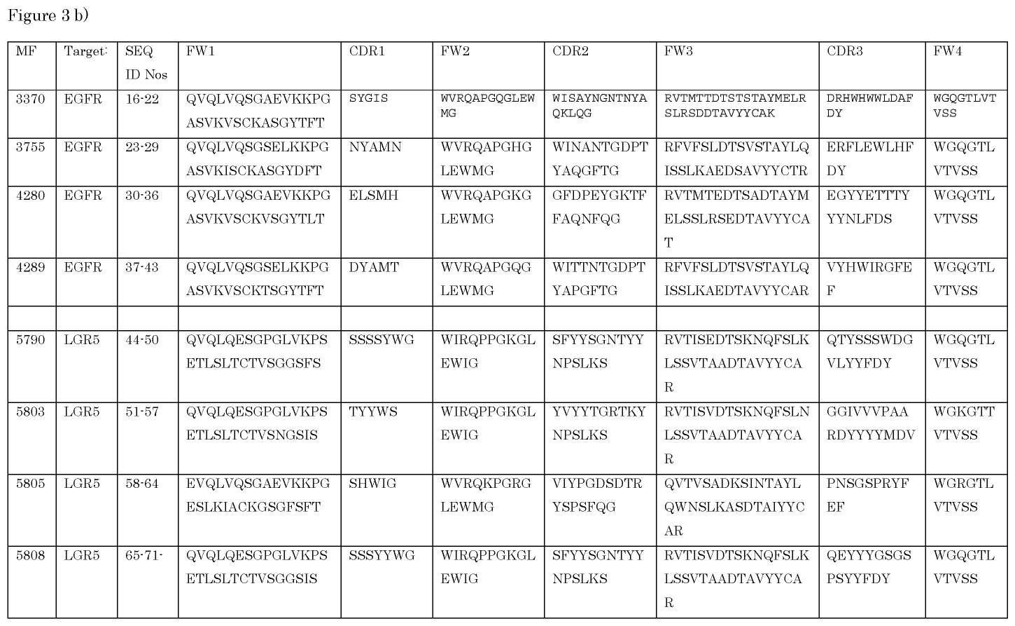

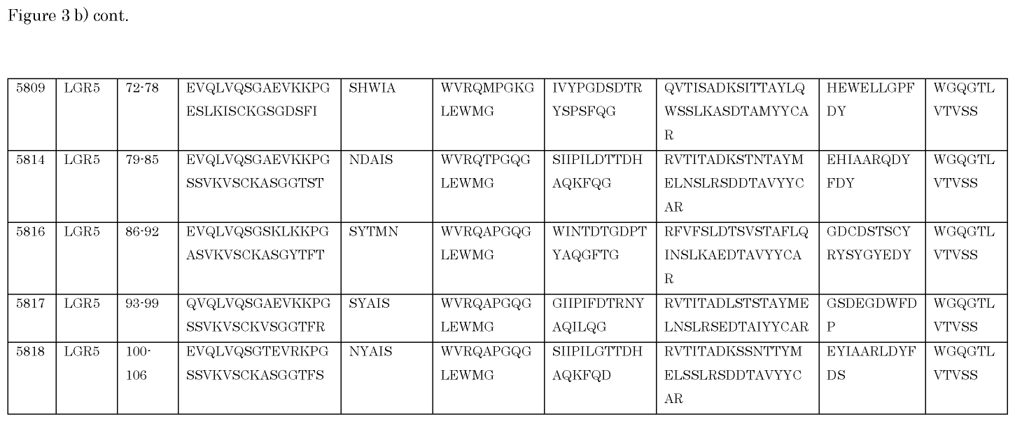

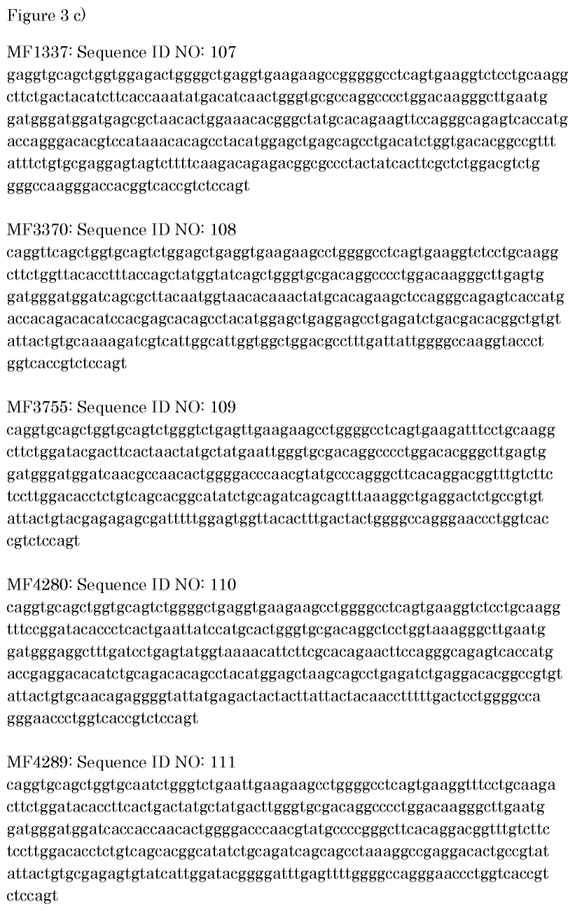

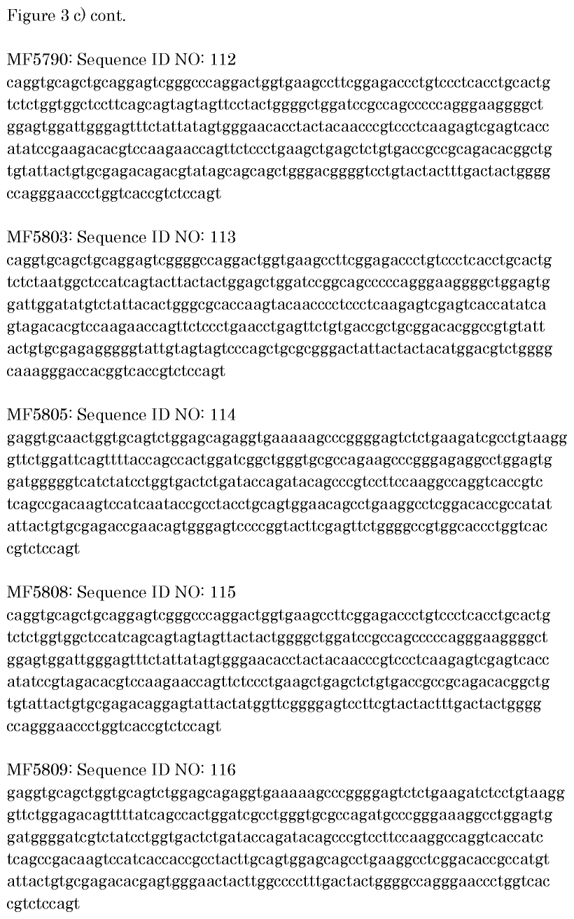

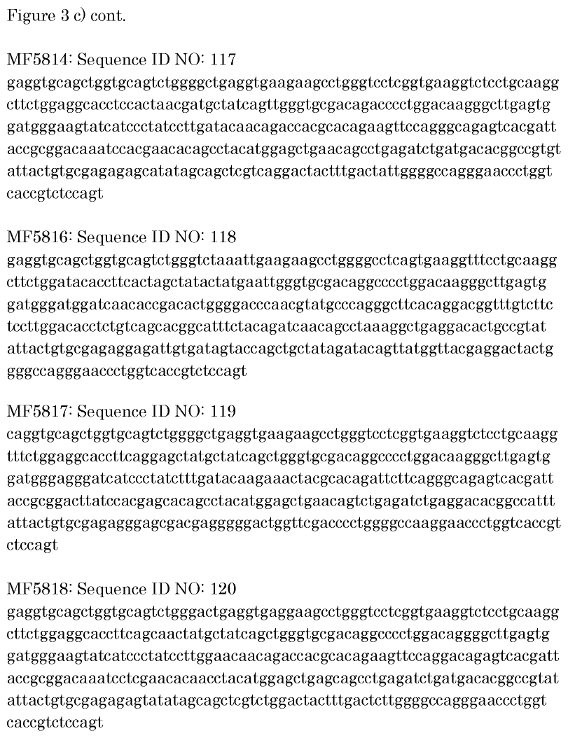

In particular, the disclosure provides an antibody or functional part, derivative and/or analogue thereof that comprises a first variable domain that binds an extracellular part of EGFR and may comprise a second variable domain that binds an extracellular part of LGR5 for use in the treatment of a gastric, esophageal, gastro-esophageal-junction or head and neck cancer, which cancer has progressed after having received prior treatment with an immune checkpoint inhibitor, wherein the subject has an Her2 status selected from being Her2 positive, Her2 high, Her2 3+, Her2 2+, Her2 1+, Her2 0 or Her2-negative subject. In certain aspects, the subject is Her2 negative. The disclosure further provides methods of treating such cancer in a Her2-negative subject, comprises providing the subject in need thereof with the antibody or functional part, derivative and/or analogue thereof. In certain aspects, said use comprises providing the subject with a flat dose of 1500 mg of the antibody or functional part, derivative and/or analogue thereof. In certain aspects, administrations of the therapeutic agent to the Her2-negative subject may be done weekly, biweekly or monthly. In certain aspects, the therapeutic agent is administered once every 2 weeks. Suitable variable domains that binds an extracellular part of EGFR and suitable variable domain that binds an extracellular part of LGR5 are disclosed herein. In certain aspects, the first variable domain comprises at least the CDR3 sequence, or at least the CDR1, CDR2 and CDR3 sequences of an EGFR specific heavy chain variable region selected from the group consisting of MF3370; MF3755; MF4280 or MF4289 as depicted in FIG. 3. In certain aspects, the second variable domain comprises at least the CDR3 sequence, or at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3.

Methods for determining an expression of human epidermal growth factor receptor 2 (HER2) of a subject are well known in the art. For instance, the expression level of Her2 can be established using immunohistochemistry (IHC) or (fluorescence) in-situ hybridization (ISH), which allows identification of a Her2-status, including identification of Her2 negative subject. IHC or ISH are both well-defined and standard procedures routinely used for establishing Her2 status in human subjects. Reference herein is made for instance to ASCO/CAP guidelines according to Bartley et al., (HER2 Testing and Clinical Decision Making in Gastroesophageal Adenocarcinoma. Arch Pathol Lab Med. 2016; 140:1345-1363). For instance, using the anti-HER-2/neu antibody (clone 4B5) allows for semi-quantitative detection of HER-2 antigen in sections of FFPE gastric, esophageal, gastro-esophageal-junction or head and neck cancer using IHC. Staining and scoring is performed according to consensus guidelines for this cancer type. Such an IHC test typically gives a score of 0 to 3+ that measures the amount of HER2 receptor protein on the surface of cells in a cancer tissue sample. Based on the IHC score, a patient can be classified as being Her2 negative, such as when a score of 0 or 1+ is measured. In case an ISH test is used to establish Her2 expression, such as using the HER2 probe (17q11.2-q12) and centromere 17 probe (Cen 17), the diagnosis is either “positive” or “negative”, sometimes also reported as “zero” for HER2. In certain aspects, a method of treatment of the present disclosure involves a subject who is Her2-negative as established by IHC and/or ISH.

With a Her2 negative subject herein is meant a subject that has a cancer, a cancer cell or a tumor, that is Her2-negative. Her2 status may be determined in accordance with IHC and/or ISH as described above.

In certain aspects, the treatment with the antibody or functional part, derivative and/or analogue thereof is preceded by a step of diagnosing the subject for Her2 status. In certain aspects, subjects having Her2-negative status are selected for treatment. In certain aspects, the treatment of a subject is preceded by a step of diagnosing a subject of having a Her2 negative gastric, esophageal, gastro-esophageal-junction or head and neck cancer. Such cancer treated by the method of the present disclosure includes gastric adenocarcinoma and esophageal cancer having squamous cell carcinoma histology.

In certain aspects, said Her2 negative diagnosis involves ISH or IHC testing of Her2 status.

In certain aspects, the treatment of a Her2 negative subject is preceded by a step of screening a subject of having a Her2 negative gastric, esophageal, or gastro-esophageal-junction cancer. Such cancer in particular is an adenocarcinoma. In certain aspects, said screening involves ISH or IHC testing of Her2 status.

In certain aspects, the subject has previously not been treated with an anti-EGFR agent. In certain aspects, the subject has not been treated with an antibody targeting EGFR. In certain aspects, the subject has not been treated with cetuximab. Such a subject is also referred to as a cetuximab-naïve or anti-EGFR-naïve subject. Worded differently, the cancer of said subject has previously not been treated with an anti-EGFR agent. In certain aspects, the cancer of said subject has not been treated with an antibody targeting EGFR. In certain aspects, the cancer of said subject has not been treated with cetuximab. Such a subject is also referred to as a cetuximab-naïve or anti-EGFR-naïve subject.

The subject of the present disclosure has received prior treatment with an immune checkpoint inhibitor. In certain aspects, the immune checkpoint inhibitor comprises durvalumab, pembrolizumab, ipilimumab, nivolumab, atezolizumab, retifanlimab cemiplimab or other anti-PD1, anti-PD-L1 antibodies approved or in development. In certain aspects, the immune checkpoint inhibitor comprises durvalumab or pembrolizumab.

Durvalumab (sold under the brand name (tradename Imfinzi™) is an FDA-approved immune checkpoint inhibitor for treating cancer, like bladder and lung cancer. It is a human immunoglobulin G1 kappa (IgG1κ) monoclonal antibody that blocks the interaction of programmed cell death ligand 1 (PD-L1) with the PD-1 (CD279). Durvalumab is an immune checkpoint inhibitor or also referred to sometimes as an immune checkpoint inhibitor drug. As shown in the example section, a clinically relevant response was observed in a patient having received prior treatment with durvalumab as immune checkpoint inhibitor.

Pembrolizumab (sold under the brand name Keytruda™), is a humanized antibody used in cancer immunotherapy to treat a variety of cancers, including melanoma, lung cancer and Hodgkin lymphoma and functions as an immune checkpoint inhibitor. It is an IgG4 isotype antibody and targets the programmed cell death protein 1 (PD-1) receptor of lymphocytes. Pembrolizumab was approved for medical use in the United States in 2014. In 2017, the US Food and Drug Administration (FDA) approved it for any unresectable or metastatic solid tumor with certain genetic anomalies. It is on the World Health Organization's List of Essential Medicines. As shown in the example section, a clinically relevant response was observed in a patient having received prior treatment with pembrolizumab as immune checkpoint inhibitor.

Ipilimumab (sold under the brand name Yervoy™), is a monoclonal antibody and immune checkpoint inhibitor that works to activate the immune system by targeting CTLA-4, a protein receptor that downregulates the immune system. Ipilimumab was approved by the US Food and Drug Administration (FDA) in March 2011, for the treatment of melanoma.

Nivolumab (sold under the brand name Opdivo™), is an immune checkpoint inhibitor used to treat a number of cancers, including melanoma, lung cancer, malignant pleural mesothelioma, renal cell carcinoma, Hodgkin lymphoma, head and neck cancer, urothelial carcinoma, colon cancer, esophageal squamous cell carcinoma, liver cancer, gastric cancer, and esophageal or gastroesophageal junction (GEJ). Nivolumab is a human IgG4 monoclonal antibody that blocks PD-1. Nivolumab was approved for medical use in the United States in 2014. It is on the World Health Organization's List of Essential Medicines. Nivolumab is the second FDA-approved systemic therapy for mesothelioma and is the first FDA-approved immunotherapy for the first-line treatment of gastric cancer.

Atezolizumab (sold under the brand name Tecentriq™), is a monoclonal antibody medication used to treat urothelial carcinoma, non-small cell lung cancer (NSCLC), triple-negative breast cancer (TNBC), small cell lung cancer (SCLC), and hepatocellular carcinoma (HCC). It is a humanized, monoclonal antibody of the IgG1 isotype and targets programmed cell death-ligand 1 (PD-L1). Atezolizumab is the first PD-L1 inhibitor approved by the U.S. Food and Drug Administration.

Retifanlimab is (previously known as MGA012) is a humanized anti-PD-1 monoclonal antibody being developed for use as monotherapy as well as in combination with other cancer therapeutics. Retifanlimab is undergoing clinical trials (NCT04472429 and NCT04205812) as a monotherapy for patients with microsatellite instability-high endometrial cancer, Merkel cell carcinoma and squamous cell carcinoma of the anal canal (SCAC); and in combination with platinum-based chemotherapy for patients with non-small cell lung cancer and SCAC. Retifanlimab has been granted orphan drug designation by the FDA for the treatment of anal cancer.

Cemiplimab (sold under the brand name Libtayo®) is a monoclonal antibody medication for the treatment of squamous cell skin cancer. Cemiplimab belongs to a class of drugs that binds to the programmed death receptor-1 (PD-1), blocking the PD-1/PD-L1 pathway. In September 2018, it was approved by the FDA for treating people with metastatic cutaneous squamous cell carcinoma (CSCC) or locally advanced CSCC who are not candidates for curative surgery or curative radiation. It was approved for medical use in the European Union in June 2019.

Also, the prior treatment with an immune checkpoint inhibitor as mentioned herein aims to target the immune checkpoint proteins comprising PD-L1, PD-1, CTLA-4, B7-1 or B7-2. Therefore, the prior treatment with an immune checkpoint inhibitor of the present disclosure targets an immune checkpoint protein selected from PD-L1, PD-1, CTLA-4, B7-1 or B7-2.

Programmed death-ligand 1 (PD-L1, CD274 or B7 homolog 1 (B7-H1); HGNC: 17635; NCBI Entrez Gene: 29126; UniProtKB/Swiss-Prot: Q9NZQ7) is a protein that in humans is encoded by the CD274 gene. This gene encodes an immune inhibitory receptor ligand that is expressed by hematopoietic and non-hematopoietic cells, such as T cells and B cells and various types of tumor cells. The encoded protein is a type I transmembrane protein that has immunoglobulin V-like and C-like domains. Interaction of this ligand with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation.

Programmed cell death protein 1 (PD-1 or CD279; HGNC: 8760; NCBI Entrez Gene: 5133; UniProtKB/Swiss-Prot: Q15116), is an immune-inhibitory receptor expressed in activated T cells; it is involved in the regulation of T-cell functions, including those of effector CD8+ T cells. In addition, this protein can also promote the differentiation of CD4+ T cells into T regulatory cells. It is expressed in many types of tumors including melanomas and has demonstrated to play a role in anti-tumor immunity. Moreover, this protein has been shown to be involved in safeguarding against autoimmunity, however, it can also contribute to the inhibition of effective anti-tumor and anti-microbial immunity

Cytotoxic T-Lymphocyte Associated Protein 4 (CTLA-4 or CD152; HGNC: 2505; NCBI Entrez Gene: 1493; UniProtKB/Swiss-Prot: P16410) is a member of the immunoglobulin superfamily and encodes a protein which transmits an inhibitory signal to T cells. The protein contains a V domain, a transmembrane domain, and a cytoplasmic tail. Alternate transcriptional splice variants, encoding different isoforms, have been characterized. The membrane-bound isoform functions as a homodimer interconnected by a disulfide bond, while the soluble isoform functions as a monomer. Mutations in this gene have been associated with insulin-dependent diabetes mellitus, Graves disease, Hashimoto thyroiditis, celiac disease, systemic lupus erythematosus, thyroid-associated orbitopathy, and other autoimmune diseases.

B7-1 or Cluster of Differentiation 80 (CD80; HGNC: 1700; NCBI Entrez Gene: 941; UniProtKB/Swiss-Prot: P33681) is a B7, type I membrane protein that is part of the immunoglobulin superfamily, with an extracellular immunoglobulin constant-like domain and a variable-like domain required for receptor binding. The protein encoded by this gene is a membrane receptor that is activated by the binding of CD28 or CTLA-4. Its functions in biological systems include induction of T-cell proliferation and cytokine production. Also, it is involved in the costimulatory signal essential for T-lymphocyte activation. T-cell proliferation and cytokine production is induced by the binding of CD28, binding to CTLA-4 has opposite effects and inhibits T-cell activation. It is closely related to CD86, another B7 protein and often works in tandem. Both CD80 and CD86 interact with costimulatory receptors CD28 and CTLA-4 CD152).

B7-2 or Cluster of Differentiation 86 (CD86; HGNC: 1705 NCBI Entrez Gene: 942 UniProtKB/Swiss-Prot: P42081) is a protein constitutively expressed on dendritic cells, Langerhans cells, macrophages, B-cells (including memory B-cells), and on other antigen-presenting cells. Along with CD80, CD86 provides costimulatory signals necessary for T cell activation and survival. Depending on the ligand bound, CD86 can signal for self-regulation and cell-cell association, or for attenuation of regulation and cell-cell disassociation. The CD86 gene encodes a type I membrane protein that is a member of the immunoglobulin superfamily. Alternative splicing results in two transcript variants encoding different isoforms.

The subject may have also been previously treated with one or more lines of standard approved therapy or standard of care. Although surgery or radiation therapy may be preferred for most patients with early or localized disease, and may be considered for locally advanced disease, it may not be possible to apply to all patients, for instance due to the anatomical location of the cancer. In certain aspects, standard approved therapy or standard of care herein includes treatment by administration of a chemotherapeutic agent, such as one or more of a platinum-based compounds (e.g. cisplatin, carboplatin), an antineoplastic compound (e.g. methotrexate), a fluoropyrimidine (e.g. fluorouracil, 5-FU, capecitabine), a taxane (e.g. docetaxel or paclitaxel) a nucleoside analogue (e.g. gemcitabine) or any combination thereof.

Thus, in certain aspects, the subject of the present disclosure has received prior treatment with a chemotherapeutic agent. In certain aspects, the chemotherapeutic agent comprises a platinum-based compound (e.g. cisplatin, carboplatin), an antineoplastic compound (e.g. methotrexate), a fluoropyrimidine (e.g. fluorouracil, 5-FU, capecitabine), a taxane (e.g. docetaxel or paclitaxel) a nucleoside analogue (e.g. gemcitabine) or any combination thereof.

According to the present disclosure, in certain aspects, the cancer and/or said subject having said cancer is wildtype for SMAD4. SMAD4 (HGNC: 6770; NCBI Entrez Gene: 4089; UniProtKB/Swiss-Prot: Q13485) belongs to the SMAD family of signal transduction proteins. SMAD proteins are phosphorylated and activated by transmembrane serine-threonine receptor kinases in response to transforming growth factor (TGF)-beta signaling. The product of this gene forms homomeric complexes and heteromeric complexes with other activated SMAD proteins, which then accumulate in the nucleus and regulate the transcription of target genes. This protein binds to DNA and recognizes an 8-bp palindromic sequence (GTCTAGAC) called the SMAD-binding element (SBE). The protein acts as a tumor suppressor and inhibits epithelial cell proliferation. It may also have an inhibitory effect on tumors by reducing angiogenesis and increasing blood vessel hyperpermeability. The encoded protein is a crucial component of the bone morphogenetic protein signaling pathway. The SMAD proteins are subject to complex regulation by post-translational modifications. Mutations or deletions in this gene have been shown to result in pancreatic cancer, juvenile polyposis syndrome, and hereditary hemorrhagic telangiectasia syndrome. Notwithstanding these previously reported implications of mutations occurring in SMAD4, the cancer of the present disclosure and/or the subject having said cancer is wildtype for SMAD4. In certain aspects, said patient or cancer does not comprise any mutations over the herein mentioned SMAD4 protein information.

Optionally, the treatment with the antibody or functional part, derivative and/or analogue thereof comprises a step of diagnosing the subject for SMAD status. In certain aspects, the treatment is preceded by a step of said diagnosing. In certain aspects, subjects having gastric cancer, esophageal cancer, gastric-esophageal-junction cancer with a wildtype SMAD4 gene and/or protein are selected for treatment. In certain aspects, the treatment of a subject is preceded by a step of diagnosing said subject of having a gastric cancer, esophageal cancer, gastric-esophageal-junction cancer characterized by a wildtype SMAD4 gene or gene product.

Cancers, such as gastric, esophageal, gastro-esophageal-junction or head and neck cancer, can be related to the presence of mutations. Such mutations include mutations in known oncogenes such as PIK3CA, KRAS, BRAF, HRAS, MAP2K1 and NOTCH1. Oncogenic mutations are generally described as activating mutations or mutations which result in new functions. Another type of cancer mutation involves tumor suppressor genes, such as TP53, MLH1, CDKN2A, and PTEN. Mutations in tumor suppressor genes are generally inactivating.

In certain aspects, the cancer has a mutation in one or more EGFR signaling pathway genes. In certain aspects, the mutation is present in a gene the expression product of which is active downstream of EGFR in the EGFR signaling pathway. In certain aspects, the cancer has a mutation in a gene, and the encoded protein, selected from AKT1, KRAS, MAP2K1, NRAS, HRAS, PIK3CA, PTEN, EGFR and/or PLCG2. In certain aspects, the cancer has a mutation in a gene coding for HRAS. In certain aspects, the cancer does not have an activating mutation in KRAS and/or BRAF.

In certain aspects, the cancer has a mutation in one or more WNT signaling pathway genes. In certain aspects, in APC, CREPPB, CUL1, EP300, SOX17 and/or TP53.

In certain aspects, the mutation in the HRAS gene is a missense mutation, a somatic mutation and/or an oncogenic driver mutation. In certain aspects, HRAS comprises mutation G12S in its protein sequence, or a G>A missense mutation leading to a G>S amino acid change. In certain aspects, missense mutation G34A in the coding sequence (CDS) of codon GGC of the HRAS gene. In certain aspects, the cancer is oral squamous cell carcinoma or squamous cell carcinoma of the buccal mucosa and comprises missense mutation G12S in HRAS.

The cancer may have a mutation in the gene coding for MAP2K1. In certain aspects, the mutation in the MAP2K1 gene is a missense mutation, a somatic mutation and/or an oncogenic driver mutation. In certain aspects, MAP2K1 comprises mutation L375R in its protein sequence, or a T>G missense mutation leading to an L>R amino acid change. In certain aspects, the missense mutation is T1124G in the coding sequence (CDS) of codon CTC of the MAP2K1 gene.

TP53 encodes a transcription factor that regulates a number of activities include stress response and cell proliferation. Mutations in TP53 are associated with various cancers and are estimated to occur in more than 50% of human cancers, including gastric and esophageal cancer. In particular, the TP53 R248Q mutation was shown to be associated with cancer, including gastric and esophageal cancer (Pitolli et al. Int. J. Mol. Sci.2019 20:6241). Nonsense mutations at positions R196 and R342 have been identified in a number of tumors such as from breast and esophagus; and ovary, prostate, breast, pancreas, stomach, colon/rectum, lung, esophagus, bone; respectively (Priestly et al. Nature 2019 575:210-216). In particular, the therapeutic agents disclosed herein are useful for treating a cancer having a TP53 mutation, in particular a mutation that results in reduced TP53 expression or activity.

MLH1 (MutL homolog 1) encodes a protein involved in DNA mismatch repair and is a known tumor suppressor gene. Mutations in MLH1 are associated with various cancers including gastrointestinal cancer. Low levels of MLH1 are also associated with esophageal cancer patients having a family history of esophageal cancer (Chang et al. Oncol Lett. 2015 9:430-436) and MLH1 is mutated in 1.39% of malignant esophageal neoplasm patients (The AACR Project GENIE Consortium. AACR Project GENIE: powering precision medicine through an international consortium. Cancer Discovery. 2017;7 (8): 818-831. Dataset Version 6). In particular, the MLH1 V384D mutation was shown to be associated with cancers, e.g., colorectal cancer (Ohsawa et al. Molecular Medicine Reports 2009 2:887-891). In certain aspects, the therapeutic agents disclosed herein are useful for treating a cancer having a MLH1 mutation, in particular a mutation which results in reduced MLH1 expression or activity.

PIK3CA (phosphatidylinositol-4,5-bisphosphate 3-kinase catalytic subunit alpha) encodes the 110 kDa catalytic subunit of PI3K (phosphatidylinositol 3-kinase). Mutations in PIK3CA are associated with various cancers include gastrointestinal cancer. As reported by the American Association for Cancer Research, PIK3CA is mutated in 12.75% of malignant solid tumor patients. In particular, the PIK3CA H1047R mutation is present in 2.91% of all malignant solid tumor patients and the PIK3CA E545K present in 2.55% of all malignant solid tumor patients (see, The AACR Project GENIE Consortium. AACR Project GENIE: powering precision medicine through an international consortium. Cancer Discovery. 2017;7 (8): 818-831. Dataset Version 6. ) In certain aspects, the therapeutic agents disclosed herein are useful for treating a cancer having a PIK3CA mutation, in particular an oncogenic mutation in PIK2CA or PIK3CA.

CDKN2A (Cyclin-dependent kinase inhibitor 2A) encodes a protein that inhibits CDK4 and ARF. As reported by the American Association for Cancer Research, CDKN2A is mutated in 22.21% of esophageal carcinoma patients, 28.7% of esophageal squamous cell carcinoma patients, and 6.08% of gastric adenocarcinoma patients. In particular, the CDKN2A W110Ter mutation is present in around 0.11% of cancer patients. (The AACR Project GENIE Consortium. AACR Project GENIE: powering precision medicine through an international consortium. Cancer Discovery. 2017;7(8): 818-831. Dataset Version 6). In certain aspects, the therapeutic agents disclosed herein are useful for treating a cancer having a CDKN2A mutation, in particular a mutation which results in reduced CDKN2A expression or activity.

PTEN (phosphatase and tensin homolog) encodes for a phosphatidylinositol 3,4,5-trisphosphate 3-phosphatase. As reported by the American Association for Cancer Research, PTEN is mutated in 6.28% of cancer patients, 3.41% of gastric adenocarcinoma patients, 2.37% of esophageal carcinoma patients, and in 2.22% of esophageal adenocarcinoma patients. In particular, the PTEN R130Ter mutation (wherein Ter refers to a termination/stop codon) is present in 0.21% of all colorectal carcinoma patients (The AACR Project GENIE Consortium. AACR Project GENIE: powering precision medicine through an international consortium. Cancer Discovery. 2017;7(8): 818-831. Dataset Version 6). In certain aspects, the therapeutic agents disclosed herein are useful for treating a cancer having a PTEN mutation, in particular a mutation which results in reduced PTEN expression or activity.

BRAF encodes serine/threonine-protein kinase B-Raf, which is involved in growth signaling. As reported by the American Association for Cancer Research, BRAF is mutated in 1.91% of gastric carcinoma patients and in in 1.93% of gastric adenocarcinoma patients. In particular, the BRAF V600E mutation is present in 2.72% of cancer patients (see, The AACR Project GENIE Consortium. AACR Project GENIE: powering precision medicine through an international consortium. Cancer Discovery. 2017;7(8): 818-831. Dataset Version 6. ). In certain aspects, the therapeutic agents disclosed herein are useful for treating a cancer having a BRAF mutation, in particular an oncogenic mutation in BRAF. However, in certain aspects, the therapeutic agents disclosed herein are useful for treating a gastric cancer that does not have BRAF mutation V600E.

KRAS (Kirsten RAt Sarcoma) encodes a protein that is party of the RAS/MAPK pathway. As reported by the American Association for Cancer Research, KRAS is mutated in 14.7% of malignant solid tumor patients with KRAS G12C present in 2.28% of all malignant solid tumor patients (see, The AACR Project GENIE Consortium. AACR Project GENIE: powering precision medicine through an international consortium. Cancer Discovery. 2017;7 (8): 818-831. Dataset Version 6. ). In certain aspects, the therapeutic agents disclosed herein are useful for treating a cancer having a KRAS mutation, in particular an oncogenic mutation in KRAS.

UGT1A1 (uridine diphosphateglucuronosyl transferase 1A1) and UGT1A8 (uridine diphosphateglucuronosyl transferase 1A8) encode enzymes of the glucuronidation pathway. Several polymorphisms which reduce enzyme activity are known to affect the metabolism and effect of irinotecan. For example, the UGT1A1*6 allele (G71R polymorphism) having an allele frequency of around 0.13% in Chinese, Korean, and Japanese populations and the UGT1A1*28 allele (dinucleotide repeat polymorphism in the TATA sequence of the promoter region) are risk factors for irinotecan induced neutropenia. In certain aspects, the therapeutic agents disclosed herein are useful for treating a cancer having a UGT1A1 and/or UGT1A8 mutation, in particular a mutation that results in reduced expression or activity of UGT1A1 and/or UGT1A8.

ATM (Ataxia Telangiectaisa Mutated), is a member of the serine-threonine kinase family and coordinates cellular responses to DNA damage through activation of distinct DNA repair and signaling pathways. ATM germline mutations are associated with ataxia telangiectasia and ATM somatic mutations are commonly observed in endometrial, colon, pancreatic, breast cancers and urothelial cancer.

Notch 1 (NOTCH1), also known as AOS5, hN1, AOVD1 and TAN1, is a gene that encodes a transmembrane protein that functions in multiple developmental processes and the interactions between adjacent cells. The transmembrane protein also functions as a receptor for membrane bound ligands. Fusions, missense mutations, nonsense mutations, silent mutations, frameshift deletions and insertions, and in-frame deletions and insertions are observed in cancers such as esophageal cancer, hematopoietic and lymphoid cancers, and stomach cancer. NOTCH1 is altered in 4.48% of all cancers with colon adenocarcinoma, lung adenocarcinoma, breast invasive ductal carcinoma, endometrial endometrioid adenocarcinoma, and skin squamous cell carcinoma having the greatest prevalence of alterations. In head and neck squamous cell carcinoma, NOTCH1 is altered in about 16% of patients (The AACR Project GENIE Consortium. Cancer Discovery. 2017;7(8): 818-831).

The HRAS (HGNC ID: 5173) gene product is involved in the activation of Ras protein signal transduction. Ras proteins bind GDP/GTP and possess intrinsic GTPase activity. Somatic mutations in the HRAS proto-oncogene have been shown to be implicated in bladder cancer, thyroid, salivary duct carcinoma, epithelial-myoepithelial carcinoma and kidney cancers (Chiosea et al., in Am. J. of Surg. Path. 39 (6): 744-52; Chiosea et al., in Head and Neck Path. 2014. 8 (2): 146-50). In some embodiments, the therapeutic compounds disclosed herein are useful for treating a cancer having an HRAS mutation, in particular an oncogenic mutation in HRAS, such as HRAS mutation G12S. The cancer in particular is HNSCC of the oral cavity or buccal mucosa.

MAP2K1 (HGNC ID: 6840) belongs to the group of mitogen-activated protein kinase kinases. It is active in MAP kinase signaling and encodes for the protein dual specificity mitogen-activated protein kinase kinase 1. As part of the MAP kinase pathway, MAP2K1 is involved in many cellular processes, including cell proliferation, differentiation, and transcriptional regulation. MAP2K1 is altered in 1.05% of all cancers with cutaneous melanoma, lung adenocarcinoma, colon adenocarcinoma, melanoma, and breast invasive ductal carcinoma having the greatest prevalence of alterations (The AACR Project GENIE Consortium. Cancer Discovery. 2017;7 (8): 818-831. Dataset Version 8). In some embodiments, the therapeutic compounds disclosed herein are useful for treating a cancer having a MAP2K1 mutation, in particular MAP2K1 mutation L375R.

In certain aspects, the disclosure provides methods for treating a cancer having a mutation in the gene encoding for TP53, MLH1, PIK3CA, CDKN2A, UGTIA, UGT1A8, BRAF, PTEN, and KRAS. In certain aspects, the cancer has one or more mutations selected from TP53 R196T; TP53 R342T; TP53 R248Q; MLH1 V384D; PIK3CA H1047R; PIK3CA E545K; CDKN2A W110T; UGT1A1 G71R; UGT1A8 G71R; and KRAS G12C. In certain aspects, the cancer is wild-type for KRAS. Alternatively, the disclosure provides methods for treating a cancer having a mutation in the gene encoding for ATM, in particular mutation W57T. In particular, the disclosure provides methods for treating esophageal cancer, in particular ESCC, having a mutation in the gene encoding for ATM, in particular mutation W57T.

In certain aspects, the cancer has a mutation in the gene coding for TP53, such as wherein the mutation is R342T, and the cancer has a mutation in the gene coding for MLH1, such as wherein the mutation is V384D. In certain aspects, the cancer has a mutation in the gene coding for TP53, In certain aspects, the mutation is R248Q. In certain aspects, the cancer has a mutation in the gene coding for PIK3CA. In certain aspects, the mutation is H1047R. In certain aspects, the cancer has a mutation in the gene coding for CDKN2A, in certain aspects, the mutation is W110T. In certain aspects, the cancer has a mutation in the gene coding for UGT1A1, in certain aspects, the mutation is G71R, and the cancer has a mutation in the gene coding for UGT1A8, in certain aspects, the mutation is G71R. In certain aspects, the cancer is esophageal cancer. In certain aspects, the cancer is esophageal squamous cell carcinoma (ESCC).

In certain aspects, the cancer has a mutation in the gene coding for BRAF. However, in certain aspects, the cancer does not have mutation V600E in the gene coding for BRAF, and in certain aspects, does not have mutation R130Ter in the gene encoding for PTEN. In certain aspects, the cancer has a mutation in the gene coding for KRAS, in certain aspects, the mutation is G12C, the cancer has a mutation in the gene coding for UGT1A1, in certain aspects, the mutation is G71R, and the cancer has a mutation in the gene coding for UGT1A8, in certain aspects, the mutation is G71R. In certain aspects, the cancer has a mutation in the gene coding for UGT1A1, in certain aspects, the mutation is G71R, and the cancer has a mutation in the gene coding for UGT1A8, in certain aspects, the mutation is G71R. In certain aspects, the cancer has a mutation in PIK3CA, in certain aspects, the mutation is E545K. In certain aspects, the cancer is gastric cancer.

In some aspects, the antibody or functional part, derivative and/or analogue thereof as disclosed herein is a multispecific antibody. In certain aspects, said antibody is a bispecific antibody. Said multi-or bispecific antibody or a functional part, derivative and/or analogue thereof, in certain aspects comprises a first variable domain that binds an extracellular part of the epidermal growth factor (EGF) receptor and a second variable domain, which in certain aspects, does not bind EGFR. In certain aspects, the antibody or functional part, derivative and/or analogue thereof binds EGFR monovalently. Also in certain aspects, said multispecific or bispecific antibody or functional part, derivative and/or analogue thereof, comprises a second variable domain that binds LGR5.

In certain aspects, the EGFR is a human EGFR. The EGFR that is bound by said antibody or functional part, derivative and/or analogue thereof of the present disclosure, includes wildtype EGFR as well as EGFR having an oncogenic driver mutation. In certain aspects, said oncogenic driver mutation is an activating EGFR mutation. In certain aspects, such a mutation does not conformationally change the epitope that is bound by the antibody of the present disclosure. In certain aspects, the EGFR mutations of the present disclosure include mutations such as exon 18 mutations, including G719A, G719C, 2E709_T710D, E709A, G719S; exon 19 deletion mutations, including deletion of LREA or VAIKEL; exon 19 point mutations G735S, P753L, L747S, D761Y; in-frame exon 20 insertion mutations of 1-7 amino acids, exon 20 point mutations, including V765A, T783A, V774A, S784P, V769M, T790M; exon 21 mutations, including L858R, T854A, A871E, L861A, L861C, L861S, V8431 or P848L. The antibody of the present disclosure binds an epitope that is not located in close proximity of said mutations. In particular, the EGFR mutation is S492R, which results in loss of binding of cetuximab to EGFR. The antibody of the present disclosure binds an epitope that is different from the epitope that is recognized by Cetuximab.

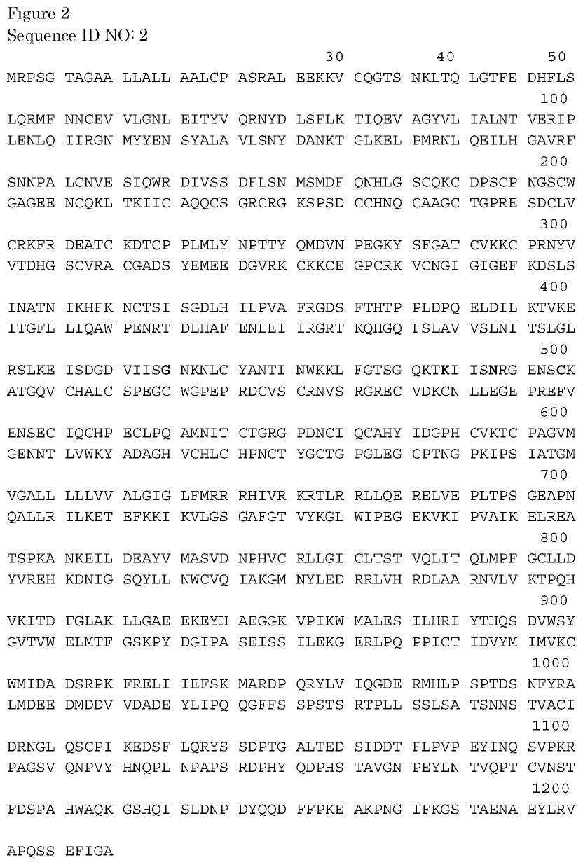

Without being bound by any theory it is believed that amino acid residues 1462; G465; K489; 1491; N493; and C499 as depicted FIG. 2 are involved in binding an epitope by an antibody of the present disclosure. In certain aspects, involvement in binding is determined by observing a reduced binding of the variable domain to an EGFR with one or more of the amino acid residue substitutions selected from 1462A; G465A; K489A; 1491A; N493A; and C499A.

In one aspect, the variable domain that binds an epitope on an extracellular part of human EGFR is a variable domain that binds an epitope that is located within amino acid residues 420-480 of the sequence depicted in FIG. 2. In certain aspects, the binding of the variable domain to EGFR is reduced by one or more of the following amino acid residue substitutions 1462A; G465A; K489A; 1491A; N493A; and C499A in EGFR. In certain aspects, binding of the antibody to human EGFR interferes with the binding of EGF to the receptor. In certain aspects, the epitope on EGFR is a conformational epitope. In one aspect, the epitope is located within amino acid residues 420-480 of the sequence depicted in FIG. 2, or within 430-480 of the sequence depicted in FIG. 2. In certain aspects, said epitope is located within 438-469 of the sequence depicted in FIG. 2.

Without being bound by theory it is believed that the contact residues of the epitope, i.e. where the variable domain contacts the human EGFR are likely 1462; K489; 1491; and N493. The amino acid residues G465 and C499 are likely indirectly involved in the binding of the antibody to EGFR.

In certain aspects, the second variable domain binds LGR5. In certain aspects, the LGR5 is a human LGR5. The multispecific or bispecific antibody or a functional part, derivative and/or analogue thereof as described herein comprises a variable domain that binds an extracellular part of a human epidermal growth factor (EGF) receptor and in certain aspects, a variable domain that binds a human LGR5.

In certain aspects, the antibody or a functional part, derivative and/or analogue thereof as described herein comprises a variable domain that binds an extracellular part of the epidermal growth factor (EGF) receptor and interferes with the binding of EGF to the receptor and a variable domain that binds LGR5 wherein interaction of the antibody with LGR5 on an LGR5-expressing cell does not block the binding of an Rspondin (RSPO) to LGR5. Methods for determining whether an antibody blocks or does not block the binding of an Rspondin to LGR5 are described in WO2017069528, which is hereby incorporated by reference.

Where herein accession numbers or alternative names of proteins/genes are given, they are primarily given to provide a further method of identification of the mentioned protein as a target, the actual sequence of the target protein bound by an antibody of the invention may vary, for instance because of a mutation and/or alternative splicing in the encoding gene such as those occurring in some cancers or the like. The target protein is bound by the antibody as long as the epitope is present in the protein and the epitope is accessible to the antibody.

In certain aspects, an antibody or a functional part, derivative and/or analogue thereof as described herein interferes with the binding of a ligand for EGFR to EGFR. The term “interferes with binding” as used herein means that binding of the antibody or a functional part, derivative and/or analogue thereof to the EGFR competes with the ligand for binding to EGF receptor. The antibody or a functional part, derivative and/or analogue thereof may diminish ligand binding, displace ligand when this is already bound to the EGF receptor or it may, for instance through steric hindrance, at least partially prevent that ligand can bind to the EGF receptor.

In certain aspects, an EGFR antibody as disclosed herein inhibits respectively EGFR ligand-induced signaling, measured as ligand-induced growth of BxPC3 cells (ATCC CRL-1687) or BxPC3-luc2 cells (Perkin Elmer 125058) or ligand-induced cell death of A431 cells (ATCC CRL-1555). EGFR can bind a number of ligands and stimulate growth of the mentioned BxPC3 cells or BxPC3-luc2 cells. In the presence of an EGFR ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100ng/ml of culture medium. In certain aspects, said EGF is the EGF R&D systems, cat. nr. 396-HB and 236-EG (see also WO2017/069628; which is incorporated by reference herein).

In certain aspects, an EGFR antibody as disclosed herein inhibits EGFR ligand induced growth of BxPC3 cells (ATCC CRL-1687) or BxPC3-luc2 cells (Perkin Elmer 125058). EGFR can bind a number of ligands and stimulate growth of the mentioned BxPC3 cells or BxPC3-luc2 cells. In the presence of a ligand the growth of BxPC3 or BxPC3-luc2 cells is stimulated. EGFR ligand-induced growth of BxPC3 cells can be measured by comparing the growth of the cells in the absence and presence of the ligand. In certain aspects, the EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100 ng/ml of culture medium. In certain aspects, EGF is the EGF of R&D systems, cat. nr. 396-HB and 236-EG (see also WO2017/069628; which is incorporated by reference herein).

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

Whether an antibody as described herein inhibits signaling or inhibits growth in a multispecific format is in certain aspects determined by the method as described herein above using a monospecific monovalent or monospecific bivalent version of the antibody. In certain aspects, such an antibody has binding sites for the receptor of which signaling is to be determined. A monospecific monovalent antibody can have a variable domain with an irrelevant binding specificity such as tetanus toxoid specificity. In certain aspects, said antibody is a bivalent monospecific antibody wherein the antigen binding variable domains consist of variable domains that bind the EGF-receptor family member.

In its Biclonics® antibody program, Merus has developed multispecific antibodies that target EGFR and LGR5 (Leucine-rich repeat containing G protein-coupled receptor). The efficacy of such multispecific antibodies has been assessed in vitro and in vivo using patient-derived CRC organoids and mice PDX models, respectively (see, e.g., WO2017/069628; which is incorporated by reference herein). Multispecific antibodies that target EGFR and LGR5 were shown to inhibit tumor growth. The potency of such inhibitory antibodies was shown to be correlated with the levels of LGR5 RNA expression by cells from the cancer. In certain aspects, said multispecific antibodies that target EGFR and LGR5 are as described in WO2017/069628.

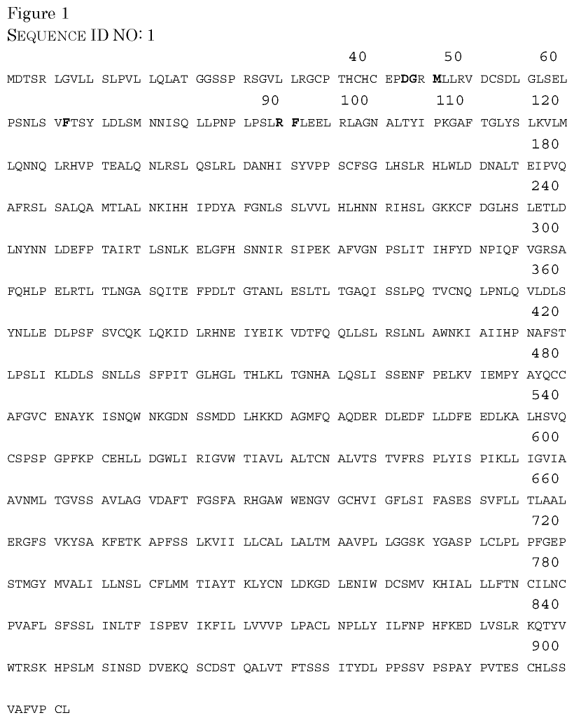

An antibody or a functional part, derivative and/or analogue thereof as described herein comprises a variable domain that binds an extracellular part of LGR5. In certain aspects, the variable domain that binds an extracellular part of LGR5 binds an epitope that is located within amino acid residues 21-118 of the sequence of FIG. 1 of which amino acid residues D43; G44, M46, F67, R90, and F91 are involved in binding of the antibody to the epitope.

In certain aspects, the LGR5 variable domain is a variable domain wherein one or more of the amino acid residue substitutions in LGR5 of D43A; G44A, M46A, F67A, R90A, and F91A reduces the binding of the variable domain to LGR5.

In certain aspects, the epitope on an extracellular part of LGR5 is located within amino acid residues 21-118 of the sequence of FIG. 1. In certain aspects, it is an epitope wherein the binding of the LGR5 variable domain to LGR5 is reduced by one or more of the following amino acid residue substitutions D43A; G44A, M46A, F67A, R90A, and F91A in LGR5.

The disclosure further provides an antibody with a variable domain that binds an extracellular part of EGFR and a variable domain that binds an extracellular part of LGR5 wherein the LGR5 variable domain binds an epitope on LGR5 that is located within amino acid residues 21-118 of the sequence of Figure. 1

In certain aspects, the epitope on LGR5 is a conformational epitope. In certain aspects, the epitope is located within amino acid residues 40-95 of the sequence of FIG. 1. In certain aspects, the binding of the antibody to LGR5 is reduced with one or more of the following amino acid residue substitutions D43A; G44A, M46A, F67A, R90A, and F91A.

Without being bound by theory it is believed that M46, F67, R90, and F91 of LGR5 as depicted in FIG. 1, are contact residues for a variable domain as indicated herein above, i.e. the antigen-binding site of a variable domain that binds the LGR5 epitope. That amino acid residue substitution D43A and G44A reduces the binding of an antibody can be due to the fact that these are also contact residues, however, it is also possible that these amino acid residue substitutions induce a (slight) modification of the conformation of the part of LGR5 that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In an exemplary method, CHO cells express LGR5 on the cell membrane, or an alanine substitution mutant, such as a mutant comprising one or more of the substitutions M46A, F67A, R90A, or F91A. A test antibody is contacted with the CHO cells and binding of the antibody to the cells compared. A test antibody binds the epitope if it binds to LGR5 and to a lesser extent to an LGR5 with a M46A, F67A, R90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein and a part that warrants association with the cell membrane of course, when cells are used. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel readily identifies whether the test antibodies exhibit reduced binding to mutants with a M46A, F67A, R90A, or F91A substitution and thus whether the test antibody is an antibody of the invention. Reduced binding to mutants with a M46A, F67A, R90A, or F91A substitution also identifies the epitope to be located within amino acid residues 21-118 of the sequence of FIG. 1. In certain aspects, the panel includes a D43A substitution mutant; a G44A substitution mutant of both. The antibody with the VH sequence of the VH of MF5816 exhibits reduced binding to these substitution mutants.

Without being bound by any theory it is believed that amino acid residues 1462; G465; K489; 1491; N493; and C499 as depicted FIG. 2 are involved in binding an epitope by an antibody comprising a variable domain as indicated herein above. In certain aspects, involvement in binding is determined by observing a reduced binding of the variable domain to an EGFR with one or more of the amino acid residue substitutions selected from 1462A; G465A; K489A; 1491A; N493A; and C499A. In an exemplary method, CHO cells express EGFR on the cell membrane, or an alanine substitution mutant, such as a mutant comprising one or more of the substitutions selected from 1462A; G465A; K489A; 1491A; N493A; and C499A. A test antibody is contacted with the CHO cells and binding of the antibody to the cells compared. A test antibody binds the epitope if it binds to EGFR and to a lesser extent to an EGFR with a 1462A; G465A; K489A; 1491A; N493A; and C499A substitution. Comparing binding with a panel of mutants each comprising one alanine residue substitution is preferred. Such binding studies are well known in the art. Often the panel comprises single alanine substitution mutants covering essentially all amino acid residues. For EGFR the panel only needs to cover the extracellular part of the protein and a part that warrants association with the cell membrane of course, when cells are used. Expression of a particular mutant can be compromised but this is easily detected by one or more EGFR antibodies that bind to different region(s). If expression is also reduced for these control antibodies the level or folding of the protein on the membrane is compromised for this particular mutant. Binding characteristics of the test antibody to the panel readily identifies whether the test antibodies exhibit reduced binding to mutants with a 1462A; G465A; K489A; 1491A; N493A; and C499A substitution.

In one aspect, the variable domain that binds an epitope on an extracellular part of human EGFR is a variable domain that binds an epitope that is located within amino acid residues 420-480 of the sequence depicted in FIG. 2. In certain aspects, the binding of the variable domain to EGFR is reduced by one or more of the following amino acid residue substitutions 1462A; G465A; K489A; 1491A; N493A; and C499A in EGFR. In certain aspects, the binding of the antibody to human EGFR interferes with the binding of EGF to the receptor. In certain aspects, the epitope on EGFR is a conformational epitope. In one aspect the epitope is located within amino acid residues 420-480 of the sequence depicted in FIG. 2, such as within 430-480 of the sequence depicted in FIG. 2. In certain aspects, said epitope is located within 438-469 of the sequence depicted in FIG. 2.

Without being bound by theory it is believed that the contact residues of the epitope, i.e. where the variable domain contacts the human EGFR are likely 1462; K489; 1491; and N493. The amino acid residues G465 and C499 are likely indirectly involved in the binding of the antibody to EGFR.

In certain aspects, the variable domain that binds human EGFR, is a variable domain with a heavy chain variable region that comprises at least the CDR3 sequence of the VH of MF3755 as depicted in FIG. 3 or a CDR3 sequence that differs in at most three, or in at most two, or in no more than one amino acid from a CDR3 sequence of the VH of MF3755 as depicted in FIG. 3.

In certain aspects, the variable domain that binds human EGFR, is a variable domain with a heavy chain variable region that comprises at least the CDR1, CDR2 and CDR3 sequences of the VH of MF3755 as depicted in FIG. 3; or the CDR1, CDR2 and CDR3 sequences of the VH of MF3755 as depicted in FIG. 3 with at most three, or at most two, or at most one amino acid substitutions.

In certain aspects, the variable domain that binds human EGFR, is a variable domain with a heavy chain variable region that comprises the sequence of the VH chain of MF3755 as depicted in FIG. 3; or the amino acid sequence of the VH chain of MF3755 depicted in FIG. 3 having at most 15 (or in certain aspects 1, 2, 3, 4, 5, 6, 7, 8, 9 or 10, or in certain aspects 1, 2, 3, 4 or 5) amino acid insertions, deletions, substitutions or a combination thereof with respect to the VH chain of MF3755.

In certain aspects, the disclosure provides an antibody comprising a variable domain that binds an extracellular part of EGFR and a variable domain that binds an extracellular part of LGR5,

wherein a heavy chain variable region of said variable domain comprises at least the CDR3 sequence of an EGFR specific heavy chain variable region selected from the group consisting of MF3370; MF3755; MF4280 or MF4289 as depicted in FIG. 3 or wherein a heavy chain variable region of said variable domain comprises a heavy chain CDR3 sequence that differs in at most three, or in at most two, or in no more than one amino acid from a CDR3 sequence of a VH selected from the group consisting of MF3370; MF3755; MF4280 or MF4289 as depicted in FIG. 3. In certain aspects, said variable domain comprises a heavy chain variable region comprising at least the CDR3sequence of MF3370; MF3755; MF4280 or MF4289 as depicted in FIG. 3.

In certain aspects, said variable domain comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 3, or heavy chain variable region comprising at least CDR1, CDR2 and CDR3 sequences that differ in at most three, or in at most two, or in at most one amino acid from the CDR1, CDR2 and CDR3 sequences of an EGFR specific heavy chain variable region selected from the group consisting of MF3370; MF3755; MF4280 or MF4289 as depicted in FIG. 3. In certain aspects, said variable domain comprises a heavy chain variable region comprising at least the CDR1, CDR2 and CDR3 sequences of MF3370; MF3755; MF4280 or MF4289 as depicted in FIG. 3. In certain aspects, the heavy chain variable region is MF3755. In certain aspects, the heavy chain variable region is MF4280.

In certain aspects, the antibody comprising a variable domain that binds an extracellular part of EGFR and a variable domain that binds an extracellular part of LGR5, wherein the EGFR binding variable domains has a CDR3, a CDR1, CDR2, and CDR3 and/or a VH sequence as indicated herein above has a variable domain that binds LGR5 that comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 3 or a heavy chain CDR3 sequence that differs in at most three, or in at most two, or in no more than one amino acid from a CDR3 sequence of a VH selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR3 sequence of MF5790; MF5803; MF 5805; MF5808; MF5809; MF5814; MF5816; MF5817; or MF5818 as depicted in FIG. 3.

In certain aspects, the LGR5 variable domain comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3, or heavy chain CDR1, CDR2 and CDR3 sequences that differ in at most three, or in at most two, or in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR1, CDR2 and CDR3sequences of MF5790; MF5803; MF5805; MF5808; MF5809; MF5814; MF5816; MF5817; or MF5818 as depicted in FIG. 3. In certain aspects, the heavy chain variable regions are MF5790; MF5803; MF5814; MF5816; MF5817; or MF5818. In certain aspects, the heavy chain variable regions are MF5790; MF5814; MF5816; and MF5818.

In certain aspects, the heavy chain variable region is MF5814, MF5818 or MF5816. In certain aspects, the heavy chain variable region is MF5816. In certain aspects, the heavy chain variable region is MF5818.

It has been shown that the antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5818 or one or more CDRs thereof.

VH chains of variable domains that bind EGFR or LGR5 can have one or more amino acid substitutions with respect to the sequence depicted in FIG. 3. In certain aspects, a VH chain has an amino acid sequence of an EGFR or LGR5 VH of FIG. 3, having at most 15, or 1, 2, 3, 4, 5, 6, 7, 8, 9 or 10 and, in certain aspects, having 1, 2, 3, 4 or 5 amino acid insertions, deletions, substitutions or a combination thereof with respect to the VH chain sequence of FIG. 3.

CDR sequences can have one or more amino acid residue substitutions with respect to a CDR sequence in the figures. Such one or more substitutions are for instance made for optimization purposes, such as to improve binding strength or the stability of the antibody. Optimization is for instance performed by mutagenesis procedures where after the stability and/or binding affinity of the resulting antibodies are preferably tested and an improved EGFR specific CDR sequence or LGR5 specific CDR sequence is preferably selected. A skilled person is well capable of generating antibody variants comprising at least one altered CDR sequence according to the invention. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

In certain aspects, the mentioned at most 15 (or in certain aspects, 1, 2, 3, 4, 5, 6, 7, 8, 9 or 10 or in certain aspects 1, 2, 3, 4 or 5) amino acid substitutions in a VH or VL as specified herein are the conservative amino acid substitutions. In certain aspects, the amino acid insertions, deletions and substitutions in a VH or VL as specified herein are not present in the CDR3 region. In certain aspects, the mentioned amino acid insertions, deletions and substitutions are also not present in the CDR1 and CDR2 regions. In certain aspects, the mentioned amino acid insertions, deletions and substitutions are also not present in the FR4 region.

In certain aspects, the mentioned at most 15 (or in certain aspects, 1, 2, 3, 4, 5, 6, 7, 8, 9 or 10, or in certain aspects 1, 2, 3, 4 or 5) amino acid substitutions are conservative amino acid substitutions. In certain aspects, the insertions, deletions, substitutions or a combination thereof are not in the CDR3 region of the VH chain, in certain aspects, not in the CDR1, CDR2 or CDR3 region of the VH chain and in certain aspects, not in the FR4 region.

An antibody comprising a variable domain that binds an extracellular part of EGFR and in certain aspects a variable domain that binds an extracellular part of LGR5 which comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5790 as depicted in FIG. 3; or
the amino acid sequence of VH chain MF5790 as depicted in FIG. 3 having at most 15(or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

An antibody comprising a variable domain that binds an extracellular part of EGFR and a variable domain that, in certain aspects, binds an extracellular part of LGR5comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5803 as depicted in FIG. 3; or
the amino acid sequence of VH chain MF5803 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

An antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5814 as depicted in FIG. 3; or
the amino acid sequence of VH chain MF5814 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

An antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5816 as depicted in FIG. 3; or
the amino acid sequence of VH chain MF5816 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

An antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5817 as depicted in FIG. 3; or
the amino acid sequence of VH chain MF5817 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

An antibody comprising a variable domain that binds an extracellular part of EGFR and a variable domain that, in certain aspects, binds an extracellular part of LGR5comprises
the amino acid sequence of VH chain MF3755 as depicted in FIG. 3 or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5818 as depicted in FIG. 3; or
the amino acid sequence of VH chain MF5818 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

Additional variants of the disclosed amino acid sequences which retain EGFR or LGR5 binding can be obtained, for example, from phage display libraries which contain the rearranged human IGKVl-39/IGKJl VL region (De Kruif et al. Biotechnol Bioeng. 2010 (106)741-50), and a collection of VH regions incorporating amino acid substitutions into the amino acid sequence of an EGFR or LGR5 VH region disclosed herein, as previously described (e.g., WO2017/069628). Phages encoding Fab regions which bind EGFR or LGR5 may be selected and analyzed by flow cytometry, and sequenced to identify variants with amino acid substitutions, insertions, deletions or additions which retain antigen binding.

The light chain variable regions of the VH/VL EGFR and LGR5 variable domains of the EGFR/LGR5 antibody may be the same or different. In certain aspects, the VL region of the VH/VL EGFR variable domain of the EGFR/LGR5 antibody is similar to the VL region of the VH/VL LGR5 variable domain. In certain aspects, VL regions in the first and second VH/VL variable domains are identical.

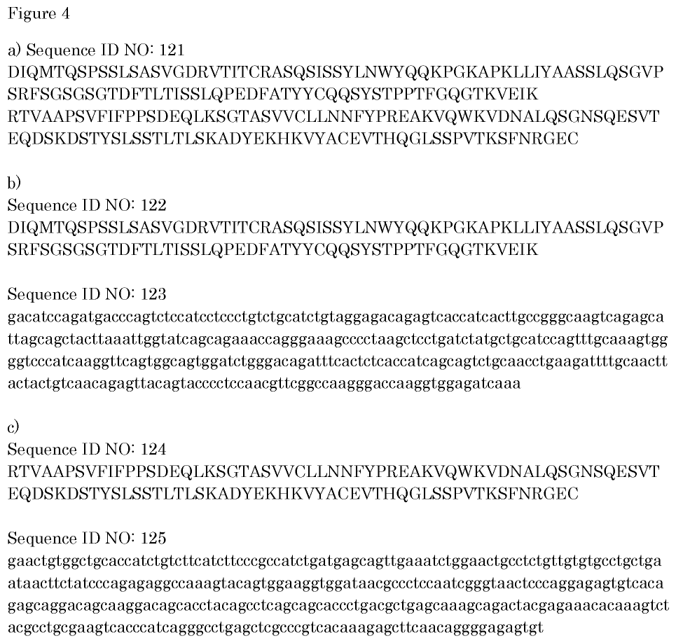

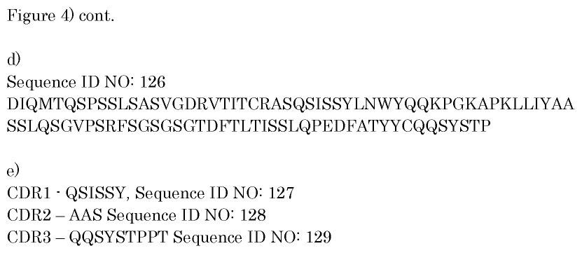

In certain aspects, the light chain variable region of one or both VH/VL variable domains of the EGFR/LGR5 antibody comprises a common light chain variable region. In certain aspects, the common light chain variable region of one or both VH/VL variable domains comprises a germline IgVκ1-39 variable region V-segment. In certain aspects, the light chain variable region of one or both VH/VL variable domains comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 4. The V-region can be combined with one of five J-regions. In certain aspects, the J-regions are jkl and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain aspects, the light chain variable region of one or both VH/VL variable domains comprises the kappa light chain IgVκ1-39*01/IGJκ1*01 or IgVκ1-39*01/IGJκ1*05 (described in FIG. 4).

In certain aspects, the light chain variable region of one or both VH/VL variable domains of the EGFR/LGR5 bispecific antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTP (described in FIG. 4) (i.e., the CDRs of IGKV1-39 according to IMGT). In certain aspects, the light chain variable region of one or both VH/VL variable domains of the EGFR/LGR5 antibody comprises an LCDR1 comprising the amino acid sequence QSISSY (described in FIG. 4), an LCDR2 comprising the amino acid sequence AASSLQS (described in FIG. 4), and an LCDR3 comprising the amino acid sequence QQSYSTP (described in FIG. 4).

In certain aspects, the light chain variable region of one or both VH/VL variable domains of the EGFR/LGR5 bispecific antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT (described in FIG. 4) (i.e., the CDRs of IGKV1-39 according to IMGT). In certain aspects, the light chain variable region of one or both VH/VL variable domains of the EGFR/LGR5 antibody comprises an LCDR1 comprising the amino acid sequence QSISSY (described in FIG. 4), an LCDR2 comprising the amino acid sequence AASSLQS (described in FIG. 4), and an LCDR3 comprising the amino acid sequence QQSYSTPPT (described in FIG. 4). Said CDR sequences are in accordance with the IMGT numbering system.

In certain aspects, one or both VH/VL variable domains of the EGFR/LGR5antibody comprise a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence of set forth in FIG. 4. In certain aspects, one or both VH/VL variable domains of the EGFR/LGR5 antibody comprise a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence of set forth in FIG. 4.

For example, the variable light chain of one or both VH/VL variable domains of the EGFR/LGR5 antibody can have from 0 to 10, or in certain aspects from 0 to 5 amino acid insertions, deletions, substitutions, additions or a combination thereof with respect to a sequence in FIG. 4. In certain aspects, the light chain variable region of one or both VH/VL variable domains of the EGFR/LGR5 antibody comprises from 0 to 9, from 0 to 8, from 0 to 7, from 0 to 6, from 0 to 5, from 0 to 4, in certain aspects from 0 to 3, in certain aspects from 0 to 2, in certain aspects from 0 to 1 and in certain aspects 0 amino acid insertions, deletions, substitutions, additions with respect to the indicated amino acid sequence, or a combination thereof.

Also, the light chain variable region of one or both VH/VL variable domains of the EGFR/LGR5 antibody may comprise the amino acid sequence of a sequence as depicted in FIG. 4. In certain aspects, both VH/VL variable domains of the EGFR/LGR5 antibody comprise identical VL regions. In certain aspects, the VL of both VH/VL variable domains of the EGFR/LGR5 bispecific antibody comprises the amino acid sequence set forth in FIG. 4. In certain aspects, the VL of both VH/VL variable domains of the EGFR/LGR5 bispecific antibody comprises the amino acid sequence set forth in FIG. 4.

In certain aspects, the EGFR/LGR5 antibody as described herein is a bispecific antibody having two variable domains, one that binds EGFR and another that binds LGR5 as described herein. EGFR/LGR5 bispecific antibodies for use in the methods disclosed herein can be provided in a number of formats. Many different formats of bispecific antibodies are known in the art, and have been reviewed by Kontermann (Drug Discov Today, 2015 July;20(7): 838-47; MAbs, 2012 March-Apr;4(2): 182-97) and in Spiess et al., (Alternative molecular formats and therapeutic applications for bispecific antibodies. Mol. Immunol. (2015) http://dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In certain aspects, the EGFR/LGR5 bispecific antibodies used in the methods provided herein are generally of the human IgG subclass (e.g., for instance IgG1, IgG2, IgG3, IgG4). In certain aspects, the antibodies are of the human IgG1 subclass. Full length IgG antibodies are preferred because of their favorable half-life and for reasons of low immunogenicity. Accordingly, the EGFR/LGR5 bispecific antibody is in certain aspects, a full length IgG molecule. In certain aspects, the EGFR/LGR5 bispecific antibody is a full length IgG1 molecule.

Accordingly, in certain aspects, the EGFR/LGR5 bispecific antibody comprises a fragment crystallizable (Fc). In certain aspects, the Fc of the EGFR/LGR5 bispecific antibody is comprised of a human constant region. A constant region or Fc of the EGFR/LGR5 bispecific antibody may contain one or more, or not more than 10, or not more than 5 amino-acid differences with a constant region of a naturally occurring human antibody. For example, each Fab-arm of the bispecific antibodies may further include an Fc-region comprising modifications promoting the formation of the bispecific antibody, promoting stability and/or other features described herein.

Bispecific antibodies are typically produced by cells that express nucleic acid(s) encoding the antibody. Accordingly, in certain aspects, the bispecific EGFR/LGR5 antibodies disclosed herein are produced by providing a cell comprising one or more nucleic acids that encode the heavy and light chain variable regions and constant regions of the bispecific EGFR/LGR5 antibody. In certain aspects, the cell is an animal cell, such as a mammal cell, or a primate cell and in certain aspects a human cell. A suitable cell is any cell capable of comprising and preferably of producing the EGFR/LGR5 bispecific antibody.

Suitable cells for antibody production are known in the art and include a hybridoma cell, a Chinese hamster ovary (CHO) cell, an NSO cell or a PER-C6 cell. Various institutions and companies have developed cell lines for the large scale production of antibodies, for instance for clinical use. Non-limiting examples of such cell lines are CHO cells, NSO cells or PER.C6 cells. In particular, said cell is a human cell. Preferably a cell is transformed by an adenovirus El region or a functional equivalent thereof. A preferred example of such a cell line is the PER.C6 cell line or equivalent thereof. In a particular, said cell is a CHO cell or a variant thereof. Preferably the variant makes use of a Glutamine synthetase (GS) vector system for expression of an antibody. In certain aspects, the cell is a CHO cell.

In certain aspects, the cell expresses the different light and heavy chains that make up the EGFR/LGR5 bispecific antibody. In certain aspects, the cell expresses two different heavy chains and at least one light chain. In certain aspects, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1 39/IGKJ1 (huVκ1 39) light chain, previously shown to be able to pair with more than one heavy chain thereby giving rise to antibodies with diverse specificities, which facilitates the generation of bispecific molecules (De Kruif et al. J. Mol. Biol. 2009 (387) 548 58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In certain aspects, the bispecific antibody of the invention comprises two different immunoglobulin heavy chains with compatible heterodimerization domains. Various compatible heterodimerization domains have been described in the art. In certain aspects, the compatible heterodimerization domains are compatible immunoglobulin heavy chain CH3 heterodimerization domains. The art describes various ways in which such hetero-dimerization of heavy chains can be achieved.

One preferred method for producing the EGFR/LGR5 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in certain aspects, the heavy chain/light chain combination that comprises the variable domain that binds EGFR, comprises a DE variant of the heavy chain. In certain aspects, the heavy chain/light chain combination that comprises the variable domain that binds LGR5 comprises a KK variant of the heavy chain.

A candidate EGFR/LGR5 IgG bispecific antibody can be tested for binding using any suitable assay. For example, binding to membrane-expressed EGFR or LGR5 on CHO cells can be assessed by flow cytometry (according to the FACS procedure as previously described in WO2017/069628). In certain aspects, the binding of a candidate EGFR/LGR5 bispecific antibody to LGR5 on CHO cells is demonstrated by flow cytometry, performed according to standard procedures known in the art. Binding to the CHO cells is compared with CHO cells that have not been transfected with expression cassettes for EGFR and/or LGR5. The binding of the candidate bispecific IgG1 to EGFR is determined using CHO cells transfected with an EGFR expression construct; a LGR5 monospecific antibody and an EGFR monospecific antibody, as well as an irrelevant IgG1 isotype control mAb are included in the assay as controls (e.g., an antibody which binds LGR5 and another antigen such as tetanus toxin (TT)).

The affinities of the LGR5 and EGFR Fabs of a candidate EGFR/LGR5 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human EGFR (Sino Biological Inc, cat. Nr. 11896-H07H) and human LGR5 protein are run over the sensor surface in a concentration range to measure on-and off-rates. After each cycle, the sensor surface is regenerated by a pulse of HCl and the bsAb is captured again. From the obtained sensorgrams, on-and off-rates and affinity values for binding to human LGR5 and EGFR are determined using the BIAevaluation software, as previously described for CD3 in US 2016/0368988.

An antibody as disclosed herein is typically a bispecific full length antibody, in certain aspects of the human IgG subclass. In certain aspects, said antibody is of the human IgG1 subclass. Such antibodies have good ADCC properties which can, if desired, be enhanced by techniques known in the art, have favorable half-life upon in vivo administration to humans and CH3 engineering technology exists that can provide for modified heavy chains that preferentially form heterodimers over homodimers upon co-expression in clonal cells.

ADCC activity of an antibody can be improved when the antibody itself has a low ADCC activity, by modifying the constant region of the antibody. Another way to improve ADCC activity of an antibody is by enzymatically interfering with the glycosylation pathway resulting in a reduced fucose. Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody as disclosed herein can be ADCC enhanced. In certain aspects, such a bispecific antibody is afucosylated. In certain aspects, a bispecific antibody comprises a reduced amount of fucosylation of the N-linked carbohydrate structure in the Fc region, when compared to the same antibody produced in a normal CHO cell. Low fucose levels are associated with increased CD16 (FcγRIIIa) binding on NK effector cells, resulting in increased ADCC activity. In certain aspects, and in addition to its direct antitumor activity, a bispecific antibody of the present disclosure can eliminate tumor cells following opsonization and subsequent natural killer (NK) cell-mediated ADCC activity and complement-dependent cytotoxic (CDC) activity.

The antibody that comprises a variable domain that binds an extracellular part of EGFR and a variable domain that binds an extracellular part of LGR5 may further comprise one or more additional variable domains that can bind one or more further targets. In certain aspects, further target is a protein, such as a membrane protein comprising an extracellular part. A membrane protein as used herein is a cell membrane protein, such as a protein that is in the outer membrane of a cell, the membrane that separates the cell from the outside world. The membrane protein has an extracellular part. A membrane protein is at least on a cell if it contains a transmembrane region that is in the cell membrane of the cell.

Antibodies with more than two variable domains are known in the art. For instance, it is possible to attach an additional variable domain. In certain aspects, an antibody with three or more variable domains is a multivalent multimer antibody as described in PCT/NL2019/050199 which is incorporated by reference herein.

In certain aspects, the antibody is a bispecific antibody comprising two variable domains, wherein one variable domain binds an extracellular part of EGFR and another variable domain binds an extracellular part of LGR5. In certain aspects, the variable domains are variable domains as described herein.

A functional part of an antibody as described herein comprises at least a variable domain that binds an extracellular part of EGFR and a variable domain that binds an extracellular part of LGR5 as described herein. It thus comprises the antigen binding parts of an antibody as described herein and typically contains the variable domains of the antibody. A variable domain of a functional part can be a single chain Fv-fragment or a so-called single domain antibody fragment. In certain aspects, the antibody parts or derivatives have at least two variable domains of an antibody or equivalents thereof. Non-limiting examples of such variable domains or equivalents thereof are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

Also provided is an antibody or functional part, derivative and/or analogue thereof as disclosed herein (i.e., the therapeutic agent) and a pharmaceutically acceptable carrier. Such pharmaceutical compositions are useful in the treatment of cancer, in particular for the treatment of gastric, esophageal, or gastro-esophageal-junction cancer. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra-articularly and the like) the active compound may be coated in a material to protect the compound from the action of acids and other natural conditions that may inactivate the compound.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage. Also included are solid preparations which are intended for conversion, shortly before use, to liquid preparations for either oral or parenteral administration. Such liquid forms include solutions, suspensions and emulsions.

The therapeutic agent disclosed can be administered according to a suitable dosage, and suitable route (e.g., intravenous, intraperitoneal, intramuscular, intrathecal or subcutaneous). For example, a single bolus may be administered, several divided doses may be administered over time or the dose may be proportionally reduced or increased as indicated by the exigencies of the therapeutic situation. In certain aspects, a subject is administered a single dose of the antibody or functional part, derivative and/or analogue thereof as disclosed herein. In certain aspects, the therapeutic agent will be administered repeatedly, over a course of treatment. For example, in certain embodiments, multiple (e.g., 2, 3, 4, 5, 6, 7, 8, 9, 10 or more) doses of the therapeutic agent are administered to a subject in need of treatment. In some embodiments, administrations of the therapeutic agent may be done weekly, biweekly or monthly.

A clinician may utilize preferred dosages as warranted by the condition of the patient being treated. The dose may depend upon a number of factors, including stage of disease, etc. Determining the specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain aspects, the therapeutic agent is administered at a dose of 0.1, 0.3, 1, 2, 3, 4, 5, 6, 7, 8, 9, or 10 mg/kg body weight. Alternatively, the therapeutic agent is administered at a dose of 0.5, 1, 2, 3, 4, 5, 6, 7, 8, 9, or 10 mg/kg body weight.

In certain aspects, the therapeutic agent is provided to a subject using a flat dosage of 1500 mg. A flat dosage offers several advantages over body-surface or weight dosing as it reduces preparation time and reduces potential dose calculation mistakes. In certain aspects, the therapeutic agent is provided at a dosage of at least 500 mg. In certain aspects, said dosage is between 1100 to 2000 mg. In certain aspects, said dosage is between 1100 to 1800 mg. As is understood by the skilled person, the dosage can be administered over time. For example, the dosage may be administered by IV, for example with a 1-6 hour infusion, preferably a 2-4 hour infusion. In certain aspects, the therapeutic agent is administered once every 2 weeks. In particular, the flat dosages disclosed herein are suitable for use in adults and/or in subjects weighing at least 35 kg. In certain aspects, the subject is afflicted with gastric, esophageal, or gastro-esophageal-junction cancer.

In certain aspects, a premedication regimen may be used. Such a regimen may be useful to reduce the likelihood or severity of an infusion-related reaction. Generally, a steroid such as dexamethasone and/or an antihistamine such as dexchlorpheniramine, diphenhydramine, or chlorpheniramine is administered (e.g., orally, intravenously) prior to antibody treatment.

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable).

With regard to the frequency of administering the therapeutic agent, one of ordinary skill in the art will be able to determine an appropriate frequency. For example, a clinician can decide to administer the therapeutic agent relatively infrequently (e.g., once every two weeks) and progressively shorten the period between doses as tolerated by the patient. Exemplary lengths of time associated with the course of therapy in accordance with the claimed method include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months;

about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

The efficacy of the treatment methods provided herein can be assessed using any suitable means. In certain aspects, the clinical efficacy of the treatment is analyzed using cancer cell number reduction as an objective response criterion. Patients, e.g., humans, treated according to the methods disclosed herein preferably experience improvement in at least one sign of cancer. In certain aspects, one or more of the following can occur: the number of cancer cells can be reduced; cancer recurrence is prevented or delayed; one or more of the symptoms associated with cancer can be relieved to some extent. In addition, in vitro assays to determine the T cell mediated target cell lysis. In certain aspects, tumor assessment is based on CT-scan and/or MRI scans, see, e.g., the RECIST 1.1 guidelines (Response Evaluation Criteria in Solid Tumours) (Eisenhauer et al., 2009 Eur J Cancer 45:228-247). Such assessments generally take place every 4-8 weeks after treatment.

In certain aspects, the tumor cells are no longer detectable following treatment as described herein. In certain aspects, a subject is in partial or full remission. In certain aspects, a subject has an increased overall survival, median survival rate, and/or progression free survival.

The therapeutic agent (i.e., an antibody or functional part, derivative and/or analogue thereof that comprises a variable domain that binds an extracellular part of EGFR and a variable domain that binds an extracellular part of LGR5) may also be used in conjunction with other well-known therapies (e.g., chemotherapy or radiation therapy) that are selected for their particular usefulness against the cancer that is being treated.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

The compounds and compositions disclosed herein are useful as therapy and in therapeutic treatments and may thus be useful as medicaments and used in a method of preparing a medicament.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate parts of the disclosure, however, it will be appreciated that the scope of the invention may include preferred aspects having combinations of all or some of the features described.

The invention is now described by reference to the following examples, which are illustrative only, and are not intended to limit the present invention. While the invention has been described in detail and with reference to specific aspects thereof, it will be apparent to one of skill in the art that various changes and modifications can be made thereto without departing from the spirit and scope thereof.

List of clauses
1. An antibody or functional part, derivative and/or analogue thereof that comprises a first variable domain that binds an extracellular part of EGFR for use in the treatment of a cancer in a subject, which cancer in said subject has progressed after having received prior treatment with an immune checkpoint inhibitor and which cancer expresses EGFR.
2. Use of an antibody or functional part, derivative and/or analogue thereof that comprises a first variable domain that binds an extracellular part of EGFR in the manufacture of a medicament for treating a cancer in a subject, which cancer in said subject has progressed after having received prior treatment with an immune checkpoint inhibitor and which cancer expresses EGFR.
3. A method of treating a subject having an EGFR expressing cancer, wherein said subject has progressed after having received prior treatment with an immune checkpoint inhibitor, the method comprising providing the subject with an effective amount of an antibody or functional part, derivative and/or analogue thereof that comprises a first variable domain that binds an extracellular part of EGFR.
4. The antibody or functional part, derivative and/or analogue thereof, or the use or the method of any one of the preceding clauses, wherein the cancer is head and neck cancer, preferably squamous cell carcinoma of the head and neck (SCCHN), and said cancer preferably expresses EGFR characterized by an IHC score of 2+ or 3+.
5. The antibody or functional part, derivative and/or analogue thereof, or the use or the method of any one of the preceding clauses, wherein the cancer is gastric, esophageal or gastric-esophageal-junction cancer having an EGFR expression characterized by an IHC score of 3+.
6. The antibody or functional part, derivative and/or analogue thereof, or the use or the method of any one of the preceding clauses, wherein the cancer is gastric, esophageal or gastric-esophageal-junction cancer having an EGFR expression characterized by an H score for EGFR of more than 200.
7. The antibody or functional part, derivative and/or analogue thereof, or the use or the method of any one of the preceding clauses, wherein the cancer is characterized by comprising an EGFR gene amplification.
8. The antibody or functional part, derivative and/or analogue thereof, or the use or the method of clause 7, wherein said EGFR gene amplification is characterized by an EGFR copy number of 8 or more as determined by next-generation sequencing on a solid tissue sample; an EGFR score of at least 2.14, or at least 2.5 as determined by next-generation sequencing on circulating tumor DNA (ctDNA); or an EGFR/CEP7 ratio of 2 or more based on FISH.
9. An antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an IHC score of 3+.
10. An antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an H score for EGFR of more than 200.
11. An antibody or functional part, derivative and/or analogue thereof that comprises a first variable domain that binds an extracellular part of EGFR for use in the treatment of a cancer in a subject, wherein the first variable domain is a heavy chain variable region that comprises
at least the CDR3 sequence of the VH of MF3370; MF3755; MF4280 or MF4289 as depicted in FIG. 3 or a CDR3 sequence that differs in at most three, preferably in at most two, preferably in no more than one amino acid from a CDR3 sequence of the VH of MF3370; MF3755; MF4280 or MF4289 as depicted in FIG. 3;
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, preferably at most two, preferably at most one amino acid substitutions; or
the sequence of the VH chain of MF3370; MF3755; MF4280 or MF4289 as depicted in FIG. 3; or the amino acid sequence of the VH chain of MF3370; MF3755; MF4280 or MF4289 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3370; MF3755; MF4280 or MF4289; and wherein said cancer is head and neck cancer, preferably squamous cell carcinoma of the head and neck (SCCHN), which cancer preferably expresses EGFR characterized by an IHC score of 2+ or 3+ or wherein said cancer is gastric, esophageal or gastric-esophageal-junction cancer having an EGFR expression characterized by an IHC score of 3+ or preferably an H score for EGFR of more than 200.
12. The antibody or functional part, derivative and/or analogue thereof, or the use or the method of any one of the preceding clauses, wherein the subject has not received prior treatment with an anti-EGFR agent.
13. The antibody or functional part, derivative and/or analogue thereof, or the use or the method of any one of clauses 1-11, wherein the subject has not received prior treatment with an antibody targeting EGFR.
14. The antibody or functional part, derivative and/or analogue thereof, or the use or the method of any one of clauses 1-11, wherein the subject has not received prior treatment with cetuximab.
15. The antibody or functional part, derivative and/or analogue thereof of any one of clauses 9-14, wherein said cancer has progressed after having received prior treatment with an immune checkpoint inhibitor.
16. The antibody or functional part, derivative and/or analogue thereof, or the use or the method of any one of the preceding clauses, wherein said cancer expresses EGFR which is characterized by an H score of between more than 200 and not more than 300.
17. The antibody or functional part, derivative and/or analogue thereof of any one of clause 16, wherein said H score for EGFR is determined using IHC.
18. The antibody or functional part, derivative and/or analogue thereof, or the use or the method of any one of the preceding clauses, wherein the subject is a mammal, preferably a human.
19. The antibody or functional part, derivative and/or analogue thereof, or the use or the method of any one of the preceding clauses, wherein said treatment comprising providing the subject with an effective amount of said antibody or functional part, derivative and/or analogue thereof.
20. The antibody or functional part, derivative and/or analogue thereof, or the use or the method of any one of the preceding clauses, wherein said treatment comprises providing a flat dose of 1500 mg of the antibody or functional part, derivative and/or analogue thereof to the subject.
29. The antibody or functional part, derivative and/or analogue thereof, or the use or the method of any one of the preceding clauses, wherein the antibody or functional part, derivative and/or analogue thereof is provided intravenously to the subject.
22. The antibody or functional part, derivative and/or analogue thereof, or the use or the method of any one of the preceding clauses, wherein the antibody or functional part, derivative and/or analogue thereof is provided weekly, biweekly or monthly, preferably biweekly, more preferably the subject is provided with at least 3 or more biweekly dosages of the antibody or functional part, derivative and/or analogue thereof.
22. The antibody or functional part, derivative and/or analogue thereof, or the use or the method of any one of the preceding clauses, wherein the antibody is ADCC enhanced.
24. The antibody or functional part, derivative and/or analogue thereof, or the use or the method of any one of the preceding clauses, wherein the antibody is afucosylated.
25. The antibody or functional part, derivative and/or analogue thereof, or the use or the method of any one of the preceding clauses, wherein the cancer is an adenocarcinoma or a squamous cell carcinoma, in particular gastric, esophageal, or gastro-esophageal-junction adenocarcinoma or in particular head and neck squamous cell carcinoma (HNSCC).
26. The antibody or functional part, derivative and/or analogue thereof, or the use or the method of any one of the preceding clauses, wherein said cancer and/or said subject is wildtype for SMAD4.
27. The antibody or functional part, derivative and/or analogue thereof, or the use or the method of any one of the preceding clauses, wherein said cancer or subject has a mutation in TP53, preferably an activating TP53 mutation.
28. The antibody or functional part, derivative and/or analogue thereof, or the use or the method of any one of the preceding clauses, wherein said cancer or subject is Her2-negative.
29. The antibody or functional part, derivative and/or analogue thereof, or the use or the method of any one of the preceding clauses, wherein the antibody is a multispecific antibody, preferably a bispecific antibody.
30. The antibody or functional part, derivative and/or analogue thereof, or the use or the method of any one of the preceding clauses, wherein the antibody comprises a second variable domain that does not bind EGFR.
31. The antibody or functional part, derivative and/or analogue thereof, or the use or the method of any one of the preceding clauses, wherein the antibody comprises a second variable domain that binds LGR5.
32. The antibody or functional part, derivative and/or analogue thereof, or the use or the method of any one of clauses 1-28, wherein the antibody is a monovalent antibody that does not comprise a second variable domain or wherein the antibody comprises said first EGFR binding variable domain as the only variable domain.
33. The antibody or functional part, derivative and/or analogue thereof, or the use or the method of any one of the preceding clauses, wherein said immune checkpoint inhibitor comprises a PD-L1 or PD-1 inhibitor.
34. The antibody or functional part, derivative and/or analogue thereof, or the use or the method of any one of the preceding clauses, wherein the treatment comprises or is preceded by a step of diagnosing the subject for EGFR status, SMAD4 status and/or Her2 status, wherein diagnosing for Her2 status is preferably by ISH or IHC.
35. The antibody or functional part, derivative and/or analogue thereof, or the use or the method of any one of the preceding clauses, wherein the first variable domain that binds EGFR binds an epitope that is located within amino acid residues 420-480 of the human EGFR sequence depicted in (FIG. 2).
36. The antibody or functional part, derivative and/or analogue thereof, or the use or the method of any one of the preceding clauses, wherein binding of the first variable domain to EGFR is reduced by one or more of the following amino acid residue substitutions 1462A; G465A; K489A; 1491A; N493A; and C499A in EGFR as compared to an EGFR protein not comprising said substitutions.
37. The antibody or functional part, derivative and/or analogue thereof, or the use or the method of any one of clauses 29-34, wherein the variable domain that binds LGR5 binds an epitope that is located within amino acid residues 21-118 of the human LGR5 sequence depicted in FIG. 1.
38. The antibody or functional part, derivative and/or analogue thereof, or the use or the method of any one of clauses 1-10 or 12-37, wherein the first variable domain is a heavy chain variable region that comprises
at least the CDR3 sequence of the VH of MF3370; MF3755; MF4280 or MF4289 as depicted in FIG. 3 or a CDR3 sequence that differs in at most three, preferably in at most two, preferably in no more than one amino acid from a CDR3 sequence of the VH of MF3370; MF3755; MF4280 or MF4289 as depicted in FIG. 3;
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, preferably at most two, preferably at most one amino acid substitutions; or
the sequence of the VH chain of MF3370; MF3755; MF4280 or MF4289 as depicted in FIG. 3; or the amino acid sequence of the VH chain of MF3370; MF3755; MF4280 or MF4289 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3370; MF3755; MF4280 or MF4289.

### EXAMPLES

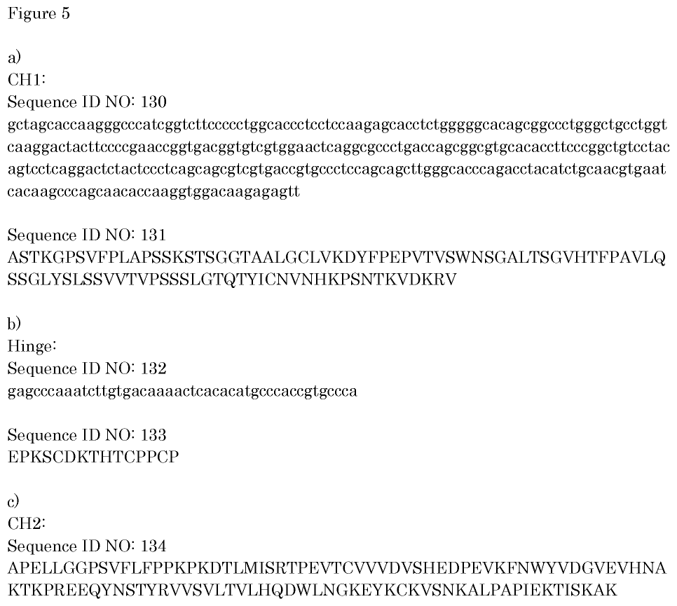

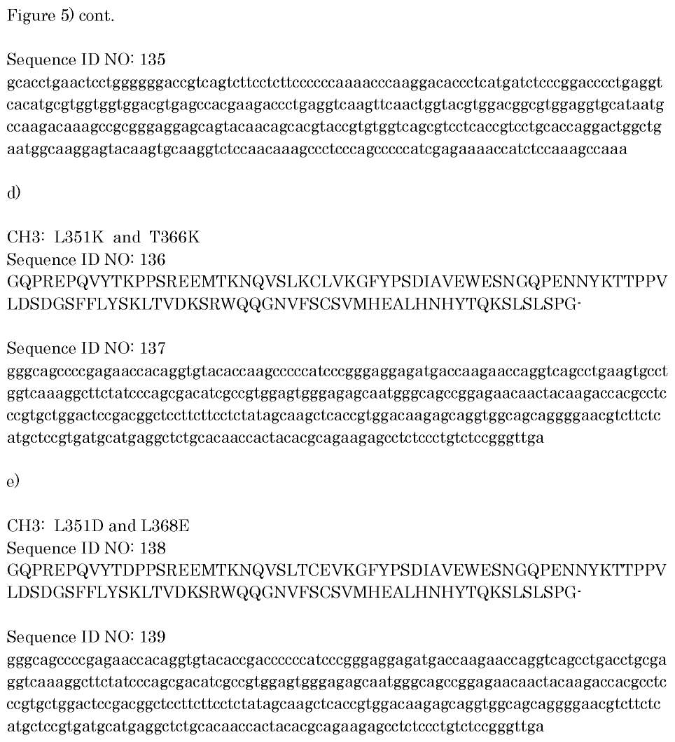

As used herein “MFXXXX” wherein X is independently a numeral 0-9, refers to a Fab comprising a variable domain wherein the VH has the amino acid sequence identified by the 4 digits depicted in FIG. 3. Unless otherwise indicated the light chain variable region of the variable domain typically has a sequence of FIG. 4b. The light chain in the examples has a sequence as depicted in FIG. 4a. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIGS. 5d and 5e. Bispecific antibodies in the examples have an Fc tail with a KK/DE CH3heterodimerization domain, a CH2 domain and a CH1 domain as indicated in FIG. 5, a common light chain as indicated in FIG. 4a and a VH as specified by the MF number. For example a bispecific antibody indicated by MF3755 xMF5816 has the above general sequences and a variable domain with a VH with the sequence of MF3755 and a variable domain with a VH with the sequence of MF5816.

The amino acid and nucleic acid sequences of the various heavy chain variable regions (VH) are indicated in FIG. 3. Bispecific antibodies EGFR/LGR5, MF3755xMF5816; comprising heavy chain variable regions MF3755 and MF5816 and a common light chain and including modifications for enhanced ADCC from afucosylation, among other LGR5 and EGFR combinations as depicted in FIG. 3 have been shown to be effective in WO2017/069628.

### Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see FIGS. 5d and 5e). It was previously demonstrated in the mentioned applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes of variable domain that bind LGR5 described above were cloned into the vector encoding the positively charged CH3 domain. The VH genes of variable domain that bind EGFR such as those disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0× 10e6 cells/ml. Cells were seeded at a density of 0.3-0.5× 10e6 viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### IgG Purification and Quantification

Purifications were performed under sterile conditions in filter plates using Protein-A affinity chromatography. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

To determine the amount of IgG purified, the concentration of antibody was determined by means of Octet analysis using protein-A biosensors (Forte-Bio, according to the supplier's recommendations) using total human IgG (Sigma Aldrich, cat. nr. 14506) as standard.

The following bispecific antibodies are suitable for use in this example and for use in the methods of the invention: MF3370xMF5790, MF3370x5803, MF3370x5805, MF3370x5808, MF3370x5809, MF3370x5814, MF3370x5816, MF3370x5817, MF3370x5818, MF3755xMF5790, MF3755x5803, MF3755x5805, MF3755x5808, MF3755x5809, MF3755x5814, MF3755x5816, MF3755x5817, MF3755x5818, MF4280xMF5790, MF4280x5803, MF4280x5805, MF4280x5808, MF4280x5809, MF4280x5814, MF4280x5816, MF4280x5817, MF4280x5818, MF4289xMF5790, MF4289x5803, MF4289x5805, MF4289x5808, MF4289x5809, MF4289x5814, MF4289x5816, MF4289x5817, and MF4289x5818. Each bispecific antibody comprises two VH as specified by the MF numbers capable of binding EGFR and LGR5 respectively, further comprises an Fc tail with a KK/DE CH3 heterodimerization domain as indicated by SEQ ID NO: 136 (FIG. 5d) and SEQ ID NO: 138 (FIG. 5e), respectively, a CH2 domain as indicated by SEQ ID NO: 134 (FIG. 5c) and a CH1 domain as indicated by SEQ ID NO: 131 (FIG. 5a), a common light chain as indicated by SEQ ID NO: 121 (FIG. 4).

### Example 1: Dose Expansion, and Efficacy of Anti-EGFRxanti-LGR5 Antibody for Patients Having EAC, GAC, GEJAC, or Head and Neck Cancer: Phase 1 dose Escalation Study in Advanced Solid Tumors

### Study Design

A phase 1 open-label multicenter study was performed with an initial dose escalation part to determine the recommended phase 2 dose (RP2D) of an anti-EGFRxanti-LGR5 bispecific antibody for solid tumors in mCRC patients with a starting dose of 5 mg flat dose. Once the RP2D is established, the antibody is further evaluated in an expansion part of the study, including in patients diagnosed with EAC, GAC, GEJAC or Head and Neck cancer, including squamous cell carcinoma of the head and neck (SCCHN). Safety, PK, immunogenicity and preliminary antitumor activity of the antibody is characterized in all patients, and biomarker analyses, including EGFR and LGR5 status is performed.

### Inclusion Criteria

Patients must fulfill all of the following requirements to enter the study:
1. Signed informed consent before initiation of any study procedures.
2. Age ≥18 years at signature of informed consent.
3. Histologically or cytologically confirmed solid tumors with evidence of metastatic or locally advanced disease not amenable to standard therapy with curative intent:

Expansion cohort: patients with advanced metastatic EAC, GAC, GEJAC head and neck squamous cell carcinoma are explored, with or without having been previously treated with at least 2 lines of standard approved therapy (when applicable).
4. A baseline fresh tumor sample (FFPE and if sufficient material also frozen) from a metastatic or primary site.
5. Amenable for biopsy.
6. Measurable disease as defined by RECIST version 1.1 by radiologic methods.
7. Eastern Cooperative Oncology Group (ECOG) performance status of 0 or 1.
8. Life expectancy ≥12 weeks, as per investigator.
9. Left ventricular ejection fraction (LVEF) ≥50% by echocardiogram (ECHO) or multiple gated acquisition scan (MUGA).
10. Adequate organ function:
Absolute neutrophil count (ANC) ≥1.5×10⁹/L
Hemoglobin ≥9 g/dL
Platelets ≥100×109/L
Corrected total serum calcium within normal ranges
Serum magnesium within normal ranges (or corrected with supplements)
Alanine aminotransferase (ALT), aspartate aminotransferase (AST) ≤2.5× upper limit of normal (ULN) and total bilirubin ≤1.5× ULN (unless due to known Gilbert's syndrome who are excluded if total bilirubin >3.0× ULN or direct bilirubin >1.5× ULN); in cases of liver involvement, ALT/AST ≤5× ULN and total bilirubin ≤2× ULN will be allowed, unless due to known Gilbert's syndrome when total bilirubin ≤3.0× ULN or direct bilirubin ≤1.5× ULN will be allowed or hepatocellular carcinoma [Child-Pugh class A] when total bilirubin <3 mg/dL will be allowed
Serum creatinine ≤1.5× ULN or creatinine clearance ≥60 mL/min calculated according to the Cockroft and Gault formula or MDRD formula for patients aged >65 years
Serum albumin >3.3 g/dL

### Exclusion Criteria

The presence of any of the following criteria excludes a patient from participating in the study:
1. Central nervous system metastases that are untreated or symptomatic, or require radiation, surgery, or continued steroid therapy to control symptoms within 14 days of study entry.
2. Known leptomeningeal involvement.
3. Participation in another clinical trial or treatment with any investigational drug within 4 weeks prior to study entry.
4. Any systemic anticancer therapy within 4 weeks or 5 half-lives whichever is longer of the first dose of study treatment. For cytotoxic agents that have major delayed toxicity (e.g. mitomycin C, nitrosoureas), or anticancer immunotherapies, a washout period of 6 weeks is required.
5. Requirement for immunosuppressive medication (e.g. methotrexate, cyclophosphamide)
6. Major surgery or radiotherapy within 3 weeks of the first dose of study treatment. Patients who received prior radiotherapy to ≥25% of bone marrow are not eligible, irrespective of when it was received.
7. Persistent grade >1 clinically significant toxicities related to prior antineoplastic therapies (except for alopecia); stable sensory neuropathy ≤grade 2 NCI-CTCAE v4.03 is allowed.
8. History of hypersensitivity reaction or any toxicity attributed to human proteins or any of the excipients that warranted permanent cessation of these agents.
9. Uncontrolled hypertension (systolic >150 mmHg and/or diastolic >100 mmHg) with appropriate treatment or unstable angina.
10. History of congestive heart failure of Class II-IV New York Heart Association (NYHA) criteria, or serious cardiac arrhythmia requiring treatment (except atrial fibrillation, paroxysmal supraventricular tachycardia).
11. History of myocardial infarction within 6 months of study entry.
12. History of prior malignancies with the exception of excised cervical intraepithelial neoplasia or non-melanoma skin cancer, or curatively treated cancer deemed at low risk for recurrence with no evidence of disease for at least 3 years.
13. Current dyspnea at rest of any origin, or other diseases requiring continuous oxygen therapy.
14. Patients with a history of interstitial lung disease (e.g.: pneumonitis or pulmonary fibrosis) or evidence of ILD on baseline chest CT scan.
15. Current serious illness or medical conditions including, but not limited to uncontrolled active infection, clinically significant pulmonary, metabolic or psychiatric disorders.
16. Active HIV, HBV, or HCV infection requiring treatment.
17. Patients with current cirrhotic status of Child-Pugh class B or C; known fibrolamellar HCC, sarcomatoid HCC, or mixed cholangiocarcinoma and HCC
18. Pregnant or lactating women; patients of childbearing potential must use highly effective contraception methods prior to study entry, for the duration of study participation, and for 6 months after the last dose of the antibody.

### Dose-Limiting Toxicity (DLT)

Any of the following clinical toxicities and/or laboratory abnormalities occurring during the first cycle (28 days) and considered by the investigator to be related to antibody treatment will be considered DLT:
Hematologic toxicities:
Grade 4 neutropenia (absolute neutrophil count [ANC] <0.5×109 cells/L) for ≥7 days
Grade 3-4 febrile neutropenia
Grade 4 thrombocytopenia
Grade 3 thrombocytopenia associated with bleeding episodes
Other grade 4 hematologic toxicity
Grade 3-4 non-hematologic AEs and laboratory toxicities with the exception of:
-Grade 3-4 infusion-related reactions
Grade 3 skin toxicity that recovers to grade ≤2 within 2 weeks with optimal treatment
Grade 3 diarrhea, nausea and/or vomiting that recover to grade ≤1 or baseline within 3 days with optimal treatment
Grade 3 electrolyte abnormalities that resolve with optimal treatment within 48 hours
Grade 3-4 liver abnormalities lasting ≤48 hours
Any liver function abnormalities that meet the definition of Hy's law.
Any drug-related toxicity lasting ≥15 days that prevents the next two administrations.

### Dose Expansion

In the expansion part, the bispecific antibody is administered at the RP2D in patients having EAC, GAC, GEJAC or head and neck cancer, in particular SCCHN. Once the RP2D has been defined, additional patients will be treated with this dose and schedule to further characterize safety, tolerability, PK and immunogenicity of antibody, and to perform a preliminary assessment of antitumor activity and biomarker evaluations.

Antibody treatment in patients with EAC, GAC, GEJAC or head and neck cancer, in particular SCCHN is explored for example, 10 to 20 patients for each indication with potential expansion up to 40 patients, conditional on signs of preliminary anti-tumor activity. The safety of the RP2D will be continuously evaluated during the expansion part of the study by the Safety Monitoring Committee. If the incidence of DLTs exceeds the predefined threshold of 33% for any cohort, enrolment will be paused for this cohort and a full review of the safety, PK, and biomarkers will be performed by the SMC in order to determine if it is safe to continue accrual in that cohort. The overall safety of the drug will also be interrogated at that time.

### Investigational Therapy and Regimen

The anti-EGFR x anti-LGR5 bispecific antibody is formulated as a clear liquid solution for IV infusion. IV infusion is performed every 2 weeks using standard infusion procedures, with a starting dose of 5 mg (flat dose), and with a recommended phase 2 dose of 1500 mg (flat dose). Dose escalation was halted once the RP2D had been reached. Infusions must be administered over a minimum of 4 hours during Cycle 1. Subsequent infusions after Cycle 1 can be reduced to 2 hours at the investigator's discretion and in the absence of IRRs. A cycle is considered 4 weeks.

### Premedication

During Cycle 1, all infusions will be administered over a period of at least 4 hours with the following premedication regimen: 24 hours before the start of the infusion, 8 mg of dexamethasone PO will be administered 1 hour before the start of the infusion, each patient will receive dexamethasone 20 mg IV, Dexchlorpheniramine 5 mg IV or diphenhydramine 50 mg PO or chlorpheniramine 10 mg IV, Ranitidine 50 mg IV or 150 mg PO and Paracetamol 1g IV or 650 mg PO.

If a patient tolerates all Cycle 1 infusions with no IRRs and the investigator considers it appropriate, the patient can continue receiving further antibody infusions without accompanying premedication of dexamethasone and the duration of infusions can be reduced to 2 hours. In such cases, the infusion duration may be extended back up to ˜4 hours where considered appropriate to avoid or reduce the incidence or severity of IRRs. For the initial antibody infusion, (Day 1 Cycle 1), each patient will be observed for 6 hours from the start of the infusion, and for 4 hours from the start of the second infusion. Thereafter patients will be observed for the duration of all subsequent administrations (a minimum of 2 hours).

### Treatment Duration

Study treatment is administered until confirmed progressive disease (as per RECIST 1.1), unacceptable toxicity, withdrawal of consent, patient non-compliance, investigator decision (e.g. clinical deterioration), or antibody interruption >6 consecutive weeks. Patients are followed up for safety for at least 30 days following the last antibody infusion and until recovery or stabilization of all related toxicities, and for disease progression and survival status for 12 months.

### Pre-Screening of Gastric Patients for EGFR Amplification or High EGFR Protein Expression

For patients with gastric/gastroesophageal junction adenocarcinoma, documentation of EGFR amplification or high EGFR expression by DNA pre-screening is required in the clinical trial. To be eligible for pre-screening, a patient must have a histological diagnosis of gastric cancer in the absence of other actionable targets. The pre-screening testing will be performed locally in a clinical laboratory improvement amendments (CLIA)-certified laboratory qualified to perform molecular screening for EGFR amplification and tumor gene mutations or EGFR IHC (e.g., EGFR PharmDx Kit or equivalent validated IVD). EGFR amplification may be tested using a FISH test, ctDNA analyses, or tissue NGS. If an appropriate local testing option is not available, samples can be sent to an approved central laboratory with the appropriate qualifications.

For ctDNA analyses, 2 tubes of 10 mL blood will be collected using tubes provided in Blood Collection Kits available from Guardant. For Guardant tissue NGS analyses FFPE slides or tissue block can be submitted using Collection Kits available from Guardant. The threshold for EGFR amplification or EGFR protein expression defined as eligible is FISH score EGFR/CEP7 ratio ≥2.0, or NGS EGFR copy ≥8, or ctDNA >2.14, or EGFR IHC H-score ≥200 (Maron SB, et al., 2018. Targeted Therapies for Targeted Populations: Anti-EGFR Treatment for EGFR-Amplified Gastroesophageal Adenocarcinoma. Cancer Discov 8:696-713.; Kato et al. 2019. Revisiting Epidermal Growth Factor Receptor (EGFR) Amplification as a Target for Anti-EGFR Therapy: Analysis of Cell-Free Circulating Tumor DNA in Patients With Advanced Malignancies. JCO Precis Oncol 3: PO.18.00180). Patients with EGFR amplification or EGFR IHC H-score ≥200 are then eligible to sign the main study ICF if they are willing and able to enter the main study. At least 10 EGFR IHC high patients will be enrolled.

Patients with documented EGFR amplification by ctDNA testing in local qualified laboratories are eligible to sign the main study ICF without the need for additional pre-screening

### Efficacy Assessments

Tumor assessment is based on CT/MRI with contrast per RECIST 1.1 (Eisenhauer et al., 2009 Eur J Cancer 45:228-247), every 8 weeks after treatment start. Objective responses must be confirmed at least 4 weeks after first observation. Bone scans are performed as clinically indicated for patients with bone metastases at baseline or suspected lesions on study. Circulating blood tumor markers, including carcinoembryonic antigen (CEA), are evaluated at screening and on Day 1 of each cycle.

### Example 2

A 67-year-old male patient with squamous cell carcinoma of the head and neck located to the larynx was enrolled in the clinical trial of Example 1. The patient had been previously treated with platinum-based chemotherapy (carboplatin), as well as paclitaxel and importantly durvalumab as immune checkpoint inhibitor.

The response as observed included a PRc of-41% after having received a bispecific antibody characterized by having first and second variable domains indicated by MF3755 xMF5816. The patient had been given more than six q2w cycles of said antibody using a 1500 mg flat dose after which the clinical response was assessed. The patient showed an EGFR IHC tumor membrane staining score of 2+.

### Example 3

A 59-year-old female patient with squamous cell carcinoma of the head and neck located in the tongue was enrolled in the clinical trial of Example 1. The patient had been previously treated with platinum-based chemotherapy (carboplatin), as well as 5-FU and importantly pembrolizumab as immune checkpoint inhibitor.

The response as observed included a partial response (PR) of-88% with a complete response (CR) at the second assessment of tumor status, after having received a bispecific antibody characterized by having first and second variable domains indicated by MF3755 xMF5816. The patient had been given more than four q2w cycles of said antibody using a 1500 mg flat dose after which the clinical response was assessed.

The patient showed an EGFR IHC tumor membrane staining score of 3+.

### Example 4

A 67-year-old male patient with squamous cell carcinoma of the head and neck of the oropharynx was enrolled in the clinical trial of Example 1. The patient had been previously treated with platinum-based chemotherapy (cisplatin and carboplatin) and, importantly pembrolizumab as immune checkpoint inhibitor.

The response as observed included a PRc of −40% after having received a bispecific antibody characterized by having first and second variable domains indicated by MF3755 xMF5816. The patient had been given eight q2w cycles of said antibody using a 1500 mg flat dose after which the clinical response was assessed.

The patient showed an EGFR IHC tumor membrane staining score of 3+.

EGFR H scoring was performed as mentioned in Example 6.

### Example 5

An 80-year-old male patient with gastric-esophageal junction cancer was enrolled in the clinical trial of Example 1. The patient had been previously treated with oxaliplatin and irinotecan-based chemotherapies.

The response as observed included a stable disease (SD) after having received a bispecific antibody characterized by having first and second variable domains indicated by MF3755 xMF5816. The patient had been given 4 q2w cycles of said antibody using a 1500 mg flat dose after which the clinical response was assessed.

The patient showed an EGFR IHC score of 3+ and an EGFR H-score of 300. Genetic profiling showed the patient was wildtype for SMAD4.

EGFR H scoring was performed as mentioned in Example 8.

### Example 6

A 62-year-old male patient with gastric cancer was enrolled in the clinical trial of Example 1. The patient had been previously treated with chemotherapy with cisplatin/capecitabine.

The response as observed included a confirmed partial response (PRc) after having received a bispecific antibody characterized by having first and second variable domains indicated by MF3755 xMF5816. The patient had been given 7 q2w cycles of said antibody using a 1500 mg flat dose after which the clinical response was assessed.

The patient showed an EGFR IHC score of 3+ and an EGFR H-score of 300. Genetic profiling showed the patient was wildtype for SMAD4.

EGFR H scoring was performed as mentioned in Example 8.

### Example 7

Safety profile at the recommenced phase 2 dose was based on 29 patients with solid tumors treated at RP2D. The most frequent adverse events were infusion-related reactions (IRR), 72% were of any grade, 7% were of grade ≥3. The time to onset: first infusion for all patients. IRRs were manageable with prophylaxis/prolonged infusion. Mild to moderate skin toxicity was observed (with 3% severe events).

Infusion-related reactions is a composite term including all AEs considered by the investigator as an IRR during 24h post-infusion.

### Example 8. EGFR Scoring via IHC

The EGFR pharmDx™ assay is a qualitative immunohistochemical (IHC) kit system to identify epidermal growth factor receptor (EGFR) expression in normal and neoplastic tissues routinely-fixed for histological evaluation. EGFR pharmDx specifically detects the EGFR (HER1) protein in EGFR-expressing cells.

The EGFR pharmDx™ assay uses the EGFR antibody, clone 2-18C9 (2-18C9) to detect EGFR protein. Clone 2-18C9 has been tested for reactivity against cell lines expressing EGFR, HER2, HER3 and HER4. In Western blots of SKBR3 and A431 cell lysates, 2-18C9 recognized a 170 kD band which is consistent with the known molecular weight of EGFR. Clone 2-18C9 has also been found to recognize the EGFRVIII (145 kD) form of the receptor in immunohistochemistry, flow cytometry and Western blotting of EGFRvIII transfected cell lines. In Western blotting experiments, 2-18C9 was unreactive with HER2 positive CAMA-1 cell lysates, HER3-transformed E. coli BL-21 protein extracts and CHO-HER4 transfected cell lysates. Additionally, Chinese Hamster Ovary (CHO) transfectants expressing myc (vector tag), either alone or coexpressed with one of the HER family members, were grown in chamber slides that were formalin-fixed and paraffin-embedded, and stained with anti-myc and 2-18C9. The myc antibody stained all five CHO transfectants, whereas 2-18C9 only stained the CHO cells transfected with HER1.

EGFR scoring is performed using the Dako EGFR pharmDx™ user protocol according to the manufacturer's instructions and recommendations. See the world wide web at agilent.com/cs/library/usermanuals/public/08052_egfr_pharmdx_interpretation_manual.p df.

### Specimen Preparation

Biopsy specimens were handled to preserve the tissue for IHC staining. Standard methods of tissue processing should be used for all specimens. Specimens preserved in the following fixatives are suitable for testing with EGFR pharmDx: 10% (v/v) neutral buffered formalin, 10% (v/v) unbuffered formalin, 25% (v/v) unbuffered formalin, AFA (acetic formalin alcohol), Richard-Allen Scientific's Pen-fix and Bouin's fixative.

### Paraffin-Embedded Sections

Routinely processed and paraffin embedded tissues are suitable for use. Specimens from the biopsy should be blocked into a thickness of 3 or 4 mm and fixed for the time period appropriate to the fixative. The tissues are then dehydrated and cleared in a series of alcohols and xylene, followed by infiltration by melted paraffin. The paraffin temperature should not exceed 60° C. Properly fixed and embedded tissue blocks expressing the EGFR protein will keep indefinitely prior to sectioning and slide mounting if stored in a cool place (15-25° C.).

Tissue specimens should be cut into sections of 3-5 μm. After sectioning, tissues should be mounted on slides and placed in drying racks. The following slides are recommended for use: Fisher's SuperFrost Plus, Dako's Silanized (code S3003), charged or poly-L-lysine coated slides. The slide racks should be pounded on an absorbent towel to remove water trapped under paraffin and on glass and then dried at room temperature for one hour. The rack of slides should then be placed in a 56-60° C. incubator for one hour. Any excess water remaining on slides after removal from the incubator should be removed by pounding slides on towels and drying for one additional hour in the incubator. After removal from the incubator, slides should be held at room temperature until cool and paraffin has hardened. To preserve antigenicity, tissue sections, mounted on slides (Fisher's SuperFrost Plus, poly-L-lysine, charged or Dako's Silanized slides (code S3003), should be stained within 2 months of sectioning when held at room temperature (20-25° C.).

The slides required for EGFR evaluation and verification of tumor presence should be prepared at the same time.

A minimum of 5 slides is recommended, 1 slide for tumor presence, 2 slides for EGFR protein evaluation (one slide for primary antibody and one slide for Negative Control Reagent), and 2 slides for back-up.

### Reagent Preparation

The following reagents are prepared prior to staining:
Wash Buffer Solution: Prepare a sufficient quantity of wash buffer by diluting Wash Buffer 10x, 1:10 using distilled or deionized water (reagent-quality water) for the wash steps. Discard buffer if cloudy in appearance.
Substrate-Chromogen Solution (DAB+): This solution should be mixed thoroughly prior to use. Any precipitate developing in the solution does not affect staining quality. To prepare DAB+Substrate-Chromogen Solution, add 11 drops of Liquid DAB+ Chromogen to one vial of DAB+ Substrate Buffer and mix. Discard any unused solution. Dilute per the guidelines above. Addition of excess Liquid DAB+ Chromogen to the DAB+Substrate Buffer will result in deterioration of the positive signal.
Counterstain. Prepare ammonia water for counterstain bluing if required.
Ammonia water (0.037 mol/L) is prepared by mixing 2.5 (±0.5) mL of 15 mol/L (concentrated) ammonium hydroxide with 1 liter of reagent quality water. Unused 0.037 mol/L ammonia water may be stored at room temperature (20-25° C.) in a tightly capped bottle for up to 12 months.
Mounting Medium. Mounting media such as Dako's Faramount Aqueous Mounting Medium, Ready-to-use (code S3025) or Dako's Glycergel Mounting Medium (code C0563) is recommended for aqueous mounting. Liquify Glycergel by warming to approximately 40(±5)° C. prior to use. Non-aqueous, permanent mounting is also suitable, such as Dako's Ultramount (code S1964)
   Staining procedure on the Dako Autostainer

### Procedural Notes

All reagents should be equilibrated to room temperature (20-25° C.) prior to immunostaining. Likewise, all incubations should be performed at room temperature.

Do not allow tissue sections to dry during the staining procedure. Dried tissue sections may display increased nonspecific staining.

Deparaffinization and Rehydration. Prior to staining, tissue slides must be deparaffinized to remove embedding medium and rehydrated. Avoid incomplete removal of paraffin. Residual embedding medium will result in increased nonspecific staining.
STEP 1. Place slides in a xylene bath and incubate for 5 (+1) minutes. Change baths and repeat once.
STEP 2. Tap off excess liquid and place slides in absolute ethanol for 3 (±1) minutes. Change baths and repeat once.
STEP 3. Tap off excess liquid and place slides in 95% ethanol for 3 (±1) minutes. Change baths and repeat once.
STEP 4. Tap off excess liquid and place slides in reagent-quality water for 5 (±1) minutes.
STEP 5. Tap off excess liquid and place slides in Wash Buffer. Begin staining procedure as outlined in Staining Protocol.

Xylene and alcohol solutions should be changed after 40 slides. Toluene or xylene substitutes, such as Histoclear, may be used in place of xylene. EGFR pharmDx includes pretreatment by means of a proteolytic enzyme digestion step. Tissue sections may occasionally be overdigested, causing disruption of cell membranes and overall tissue architecture. Run the assay with careful attention to the duration of the proteolytic digestion step.

### Post-Fixation Procedure

1. Deparaffinize sections and immerse in reagent quality water.
2. Immerse slides in a 10% neutral buffered formalin for 10 minutes.
3. Rinse slides twice in deionized or distilled water.
4. Continue with the EGFR pharmDx staining procedure.

### Automated Staining Protocol

STEP 1. Select desired protocol and program staining run.
STEP 2. Use Auto programs to set up program and begin the EGFR pharmDx program.
STEP 3. Place the reagent vials in the DAKO Autostainer reagent rack according to the computer generated reagent map.
STEP 4. Load the slides onto the DAKO Autostainer according to the computer generated slide map.
STEP 5. Begin the run.
STEP 6. Remove slides from the DAKO Autostainer.

Proceed to Counterstain and Mounting. Rinse slides in reagent-quality water after the DAB+ Substrate-Chromogen solution step. (DAKO Autostainer hardware versions 02 and 03 rinse the slides in reagent-quality water after the substrate-chromogen step. The 01 hardware version of the DAKO Autostainer rinses slides in buffer. Therefore, slides that are stained on 01 hardware must be rinsed with the reagent-quality water after they have been removed from the Autostainer).

### Interpretation of Staining Procedure

Slide evaluation should be performed by a pathologist using a light microscope. All assessments are to be made on the tumor region of the specimen. For evaluation of the immunocytochemical staining and scoring, an objective of 10X or 20X magnification is appropriate.

Use intact cells for interpretation of staining results; necrotic or degenerated cells often stain nonspecifically. Positive and negative cell lines are included in each EGFR pharmDx kit to validate staining runs, every time the assay is performed. Appropriate staining of the control cell lines provides evidence that the EGFR pharmDx assay is functioning properly. No membrane staining of the CAMA-1 control cell line (0) and moderate brown complete or incomplete membrane staining in the HT-29 control cell line (2+) indicates that the staining run is valid. If the staining intensity of the positive control cell line is too weak or too strong a false negative or false positive result may be obtained and the test should be repeated. Reference images are available in the EGFR pharmDx Interpretation Guide.

EGFR pharmDx primarily stains cell membranes, demonstrating both complete and incomplete circumferential staining. The immunostaining pattern is frequently heterogeneous, exhibiting various staining intensities within a single neoplasm. Staining has also been observed in the cytoplasm and extracellular spaces. Cytoplasmic staining is commonly seen, however the test should be repeated if significant cytoplasmic staining makes it difficult to distinguish membrane staining and interpret the results.

Tumors should be reported as EGFR-positive or EGFR-negative using membrane staining as the evaluable structure. A tumor cell is EGFR-positive if it possesses any membrane staining above background, whether or not it is completely circumferential. A tumor with no membrane staining above background in any tumor cell is reported as an EGFR-negative tumor.

Depending on the incubation length and potency of the hematoxylin used, counterstaining will result in a pale to dark blue coloration of the cell nuclei. Excessive or incomplete counterstaining may compromise interpretation of results.

Staining intensity is established as follows: 3+ (strong staining): visible at low levels of magnification, x5 objective lens which could be confirmed at higher levels as required; 2+ (moderate staining): visible at intermediate levels of magnification, x10 or x20 objective lenses; 1+ (weak staining): only reliably confirmable at high magnification, x40 objective lens; 0 (no staining): no staining visible at high magnification.

| Report to | | |
|---|---|---|
| treating physician | Definition | |
| EGFR negative | Absence of membrane staining above background in | |
| tumor | all tumor cells | |
| EGFR positive | EGFR-positive staining is defined as any IHC | |
| tumor | staining of tumor cell membranes above background | |
| | level; whether it is complete or incomplete | |
| | circumferential staining | |
| | | Percent of tumor |
| | Staining intensity | cells staining |
| | 1+, 2+ or 3+ | >0% |

### EGFR H-Scoring

Assessment of membranous staining using IHC classifies samples into 4 staining intensity categories (0 to 3+). Of note is that only linear intercellular staining of tumor cells is considered as positive and complete and incomplete membranous staining is considered and recorded. Also, for Histo-score calculation all membrane staining is considered independent of the completeness (complete and incomplete membranous staining).

H-score is assigned using the following formula: [1×(% cells having 1+ staining)+2×(% cells having 2+ staining)+3×(% cells having 3+ staining)] resulting in an H-score for EGFR between 0-300.

1. An antibody or functional part, derivative and/or analogue thereof that comprises a first variable domain that binds an extracellular part of EGFR for use in the treatment of a cancer in a subject, which cancer in said subject has progressed after having received prior treatment with an immune checkpoint inhibitor and which cancer expresses EGFR.

2. Use of an antibody or functional part, derivative and/or analogue thereof that comprises a first variable domain that binds an extracellular part of EGFR in the manufacture of a medicament for treating a cancer in a subject, which cancer in said subject has progressed after having received prior treatment with an immune checkpoint inhibitor and which cancer expresses EGFR.

3. A method of treating a subject having an EGFR expressing cancer, wherein said subject has progressed after having received prior treatment with an immune checkpoint inhibitor, the method comprising providing the subject with an effective amount of an antibody or functional part, derivative and/or analogue thereof that comprises a first variable domain that binds an extracellular part of EGFR.

4. The antibody or functional part, derivative and/or analogue thereof, or the use or the method of any one of the preceding claims, wherein the cancer is head and neck cancer, preferably squamous cell carcinoma of the head and neck (SCCHN), and said cancer preferably expresses EGFR characterized by an IHC score of 2+ or 3+.

5. The antibody or functional part, derivative and/or analogue thereof, or the use or the method of any one of the preceding claims, wherein the cancer is gastric, esophageal or gastric-esophageal-junction cancer having an EGFR expression characterized by an IHC score of 3+.

6. The antibody or functional part, derivative and/or analogue thereof, or the use or the method of any one of the preceding claims, wherein the cancer is gastric, esophageal or gastric-esophageal-junction cancer having an EGFR expression characterized by an H score for EGFR of more than 200.

7. An antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an IHC score of 3+.

8. An antibody or functional part, derivative and/or analogue thereof that comprises a first variable domain that binds an extracellular part of EGFR for use in the treatment of gastric, esophageal or gastric-esophageal-junction cancer in a subject, wherein said cancer expresses EGFR which is characterized by an H score for EGFR of more than 200.

9. An antibody or functional part, derivative and/or analogue thereof that comprises a first variable domain that binds an extracellular part of EGFR for use in the treatment of a cancer in a subject, wherein the first variable domain is a heavy chain variable region that comprises
at least the CDR3 sequence of the VH of MF3370; MF3755; MF4280 or MF4289 as depicted in FIG. 3 or a CDR3 sequence that differs in at most three, preferably in at most two, preferably in no more than one amino acid from a CDR3 sequence of the VH of MF3370; MF3755; MF4280 or MF4289 as depicted in FIG. 3;
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, preferably at most two, preferably at most one amino acid substitutions; or
the sequence of the VH chain of MF3370; MF3755; MF4280 or MF4289 as depicted in FIG. 3; or the amino acid sequence of the VH chain of MF3370; MF3755; MF4280 or MF4289 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3370; MF3755; MF4280 or MF4289; and wherein said cancer is head and neck cancer, preferably squamous cell carcinoma of the head and neck (SCCHN), which cancer preferably expresses EGFR characterized by an IHC score of 2+ or 3+ or wherein said cancer is gastric, esophageal or gastric-esophageal-junction cancer having an EGFR expression characterized by an IHC score of 3+ or preferably an H score for EGFR of more than 200.

10. The antibody or functional part, derivative and/or analogue thereof, or the use or the method of any one of the preceding claims, wherein the subject has not received prior treatment with an anti-EGFR agent.

11. The antibody or functional part, derivative and/or analogue thereof, or the use or the method of any one of claims 1-9, wherein the subject has not received prior treatment with an antibody targeting EGFR.

12. The antibody or functional part, derivative and/or analogue thereof, or the use or the method of any one of claims 1-9, wherein the subject has not received prior treatment with cetuximab.

13. The antibody or functional part, derivative and/or analogue thereof of any one of claims 7-12, wherein said cancer has progressed after having received prior treatment with an immune checkpoint inhibitor.

14. The antibody or functional part, derivative and/or analogue thereof, or the use or the method of any one of the preceding claims, wherein said cancer expresses EGFR which is characterized by an H score of between more than 200 and not more than 300.

15. The antibody or functional part, derivative and/or analogue thereof of claim 14, wherein said H score for EGFR is determined using IHC.

16. The antibody or functional part, derivative and/or analogue thereof, or the use or the method of any one of the preceding claims, wherein the subject is a mammal, preferably a human.

17. The antibody or functional part, derivative and/or analogue thereof, or the use or the method of any one of the preceding claims, wherein said treatment comprising providing the subject with an effective amount of said antibody or functional part, derivative and/or analogue thereof.

18. The antibody or functional part, derivative and/or analogue thereof, or the use or the method of any one of the preceding claims, wherein said treatment comprises providing a flat dose of 1500 mg of the antibody or functional part, derivative and/or analogue thereof to the subject.

19. The antibody or functional part, derivative and/or analogue thereof, or the use or the method of any one of the preceding claims, wherein the antibody or functional part, derivative and/or analogue thereof is provided intravenously to the subject.

20. The antibody or functional part, derivative and/or analogue thereof, or the use or the method of any one of the preceding claims, wherein the antibody or functional part, derivative and/or analogue thereof is provided weekly, biweekly or monthly, preferably biweekly, more preferably the subject is provided with at least 3 or more biweekly dosages of the antibody or functional part, derivative and/or analogue thereof.

21. The antibody or functional part, derivative and/or analogue thereof, or the use or the method of any one of the preceding claims, wherein the antibody is ADCC enhanced.

22. The antibody or functional part, derivative and/or analogue thereof, or the use or the method of any one of the preceding claims, wherein the antibody is afucosylated.

23. The antibody or functional part, derivative and/or analogue thereof, or the use or the method of any one of the preceding claims, wherein the cancer is an adenocarcinoma or a squamous cell carcinoma, in particular gastric, esophageal, or gastro-esophageal-junction adenocarcinoma or in particular head and neck squamous cell carcinoma (HNSCC).

24. The antibody or functional part, derivative and/or analogue thereof, or the use or the method of any one of the preceding claims, wherein said cancer and/or said subject is wildtype for SMAD4.

25. The antibody or functional part, derivative and/or analogue thereof, or the use or the method of any one of the preceding claims, wherein said cancer or subject has a mutation in TP53, preferably an activating TP53 mutation.

26. The antibody or functional part, derivative and/or analogue thereof, or the use or the method of any one of the preceding claims, wherein said cancer or subject is Her2-negative.

27. The antibody or functional part, derivative and/or analogue thereof, or the use or the method of any one of the preceding claims, wherein the antibody is a multispecific antibody, preferably a bispecific antibody.

28. The antibody or functional part, derivative and/or analogue thereof, or the use or the method of any one of the preceding claims, wherein the antibody comprises a second variable domain that does not bind EGFR.

29. The antibody or functional part, derivative and/or analogue thereof, or the use or the method of any one of the preceding claims, wherein the antibody comprises a second variable domain that binds LGR5.

30. The antibody or functional part, derivative and/or analogue thereof, or the use or the method of any one of claims 1-26, wherein the antibody is a monovalent antibody that does not comprise a second variable domain or wherein the antibody comprises said first EGFR binding variable domain as the only variable domain.

31. The antibody or functional part, derivative and/or analogue thereof, or the use or the method of any one of the preceding claims, wherein said immune checkpoint inhibitor comprises a PD-L1 or PD-1 inhibitor.

32. The antibody or functional part, derivative and/or analogue thereof, or the use or the method of any one of the preceding claims, wherein the treatment comprises or is preceded by a step of diagnosing the subject for EGFR status, SMAD4 status and/or Her2 status, wherein diagnosing for Her2 status is preferably by ISH or IHC.

33. The antibody or functional part, derivative and/or analogue thereof, or the use or the method of any one of the preceding claims, wherein the first variable domain that binds EGFR binds an epitope that is located within amino acid residues 420-480 of the human EGFR sequence depicted in FIG. 2.

34. The antibody or functional part, derivative and/or analogue thereof, or the use or the method of any one of the preceding claims, wherein binding of the first variable domain to EGFR is reduced by one or more of the following amino acid residue substitutions I462A; G465A; K489A; I491A; N493A; and C499A in EGFR as compared to an EGFR protein not comprising said substitutions.

35. The antibody or functional part, derivative and/or analogue thereof, or the use or the method of any one of claims 29-34, wherein the variable domain that binds LGR5 binds an epitope that is located within amino acid residues 21-118 of the human LGR5 sequence depicted in FIG. 1.

36. The antibody or functional part, derivative and/or analogue thereof, or the use or the method of any one of claim 1-8 or 10-35, wherein the first variable domain is a heavy chain variable region that comprises
at least the CDR3 sequence of the VH of MF3370; MF3755; MF4280 or MF4289 as depicted in FIG. 3 or a CDR3 sequence that differs in at most three, preferably in at most two, preferably in no more than one amino acid from a CDR3 sequence of the VH of MF3370; MF3755; MF4280 or MF4289 as depicted in FIG. 3;
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, preferably at most two, preferably at most one amino acid substitutions; or
the sequence of the VH chain of MF3370; MF3755; MF4280 or MF4289 as depicted in FIG. 3; or the amino acid sequence of the VH chain of MF3370; MF3755; MF4280 or MF4289 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3370; MF3755; MF4280 or MF4289.
